(19) United States
(12) Patent Application Publication
(10) Pub. No.: US 2022/0389380 A1
(43) Pub. Date: Dec. 8, 2022

(54) ACOUSTIC SEPARATION FOR HIGH-SPECIFICITY PURIFICATION

(71) Applicant: THE CHARLES STARK DRAPER LABORATORY, INC., Cambridge, MA (US)

(72) Inventor: Jason O. Fiering, Boston, MA (US)

(21) Appl. No.: 17/834,652

(22) Filed: Jun. 7, 2022

Related U.S. Application Data
(60) Provisional application No. 63/197,971, filed on Jun. 7, 2021.

(51) Int. Cl. C12N 5/078; C12N 5/00; B01L 3/00

(52) U.S. Cl. CPC C12N 5/0634; C12N 5/0081; B01L 3/502761; B01L 2200/0652; B01L 2400/0436; B01L 2200/0636; B01L 2400/0439; B01L 2200/16

(57) ABSTRACT

A method for separating cells in a biofluid includes pretreating the biofluid by introducing a predetermined amount of a cocktail of antibodies, flowing the pretreated biofluid through a microfluidic separation channel, and applying acoustic energy to the pretreated biofluid within the microfluidic separation channel. A system for microfluidic cell separation, capable of separating target cells from non-target cells in a biofluid includes at least one microfluidic separation channel, a source of biofluid, a source of an additive including the cocktail of antibodies, and at least one acoustic transducer coupled to the microfluidic separation channel. A kit for microfluidic cell separation is also disclosed. A method of facilitating separation of cells is also disclosed.

### CROSS-REFERENCE TO RELATED APPLICATIONS

This application claims priority under 35 U.S.C. § 119(e) to U.S. Provisional Patent Application Ser. No. 63/197,971, titled “ACOUSTIC SEPARATION FOR BIOPROCESSING,” filed Jun. 7, 2021, which is incorporated by reference herein in its entirety for all purposes.

### STATEMENT REGARDING FEDERALLY SPONSORED RESEARCH

This invention was made with government support under Grant No. 75F40119C10122 awarded by the U.S. Food and Drug Administration (FDA). The government has certain rights in the invention.

### FIELD OF TECHNOLOGY

Aspects and embodiments disclosed herein relate to systems and methods for the separation of cells. In particular, aspects and embodiments disclosed herein relate to systems and methods for the separation of target cells in a biofluid from non-target cells in the biofluid.

### SUMMARY

In accordance with one aspect, there is provided a method of separating target cells from non-target cells in a biofluid. The method may comprise pretreating the biofluid by introducing into the biofluid a predetermined amount of a cocktail of bifunctional antibodies selected to bind the non-target cells to form non-target cell clusters, producing a pretreated biofluid comprising the target cells and the non-target cell clusters. The method may comprise flowing the pretreated biofluid into an inlet of a microfluidic separation channel. The method may comprise applying acoustic energy to the pretreated biofluid within the microfluidic separation channel, such that the target cells accumulate within at least one primary stream along the separation channel and the non-target cell clusters accumulate within at least one secondary stream along the separation channel.

In some embodiments, the bifunctional antibodies comprise at least one binding site having a non-specific affinity.

In some embodiments, pretreating the biofluid comprises introducing into the biofluid the predetermined amount of the cocktail of bifunctional antibodies without a capture particle.

The method may further comprise selecting the biofluid from blood buffy coat, leukapheresis product, peripheral blood, whole blood, lymph fluid, synovial fluid, spinal fluid, bone marrow, ascities fluid, and combinations or subcomponents thereof.

The method may further comprise selecting the target cells to be leukocytes selected from the group consisting of mononuclear cells, lymphocytes, monocytes, granulocytes, agranulocytes, macrophages, T cells, B cells, NK cells, subclasses thereof, and combinations thereof.

In some embodiments, the non-target cells comprise leukocytes selected from the group consisting of mononuclear cells, lymphocytes, monocytes, granulocytes, agranulocytes, macrophages, T cells, B cells, NK cells, subclasses thereof, and combinations thereof, other than the selected target cells.

The method may comprise selecting the target cells to be lymphocytes.

The method may further comprise selecting or designing the cocktail of the bifunctional antibodies responsive to a measured or expected cell population of the biofluid.

In some embodiments, the bifunctional antibodies have at least one binding site having affinity for a cluster-forming cell.

In some embodiments, the cluster-forming cells comprise erythrocytes or platelets.

The method may further comprise controlling a ratio of the cluster-forming cells to the non-target cells in the biofluid.

The method may further comprise introducing into the biofluid at least some of the cluster-forming cells.

The method may further comprise obtaining the biofluid from a donor subject.

The method may further comprise post-treating the at least one primary stream.

The method may further comprise introducing the post-treated primary stream into a recipient subject.

In some embodiments, the method may further comprise flowing a second fluid adjacent to the biofluid into an inlet of the microfluidic separation channel, such that the biofluid and the second fluid flow in substantially parallel, substantially laminar flow.

In some embodiments, the method may further comprise flowing the pretreated biofluid into the inlet of the microfluidic separation channel at a flow rate of between about 0.03 mL/min to about 0.5 mL/min.

The method may further comprise selecting the predetermined amount responsive to a parameter selected from input biofluid load, concentration of the target cells, concentration of the non-target cells, or concentration of cluster-forming cells of the biofluid.

In some embodiments, the method may further comprise measuring at least one of the input biofluid load, concentration of the target cells, concentration of the non-target cells, and concentration of the cluster-forming cells of the biofluid prior to pretreating the biofluid.

The method may further comprise selecting a flow rate of the pretreated biofluid responsive to a parameter selected from pressure and acoustic energy within the microfluidic separation channel.

The method may further comprise measuring at least one of pressure and acoustic energy within the microfluidic separation channel.

In accordance with another aspect, there is provided a system for microfluidic cell separation configured to separate target cells from non-target cells in a biofluid. The system may comprise at least one microfluidic separation channel comprising at least one inlet, a first outlet, and a second outlet. The system may comprise a source of the biofluid in fluid communication with the at least one inlet of the at least one microfluidic separation channel. The system may comprise a source of an additive in fluid communication with the source of the biofluid, the additive comprising a cocktail of bifunctional antibodies selected to bind the non-target cells to form non-target cell clusters, producing a pretreated biofluid comprising the target cells and the non-target cell clusters. The system may comprise at least one acoustic transducer coupled to a wall of the at least one microfluidic separation channel.

In some embodiments, the additive is substantially free of capture particles.

In some embodiments, the system may further comprise a control module configured to introduce a predetermined volume of the additive into the biofluid to produce the pretreated biofluid.

In some embodiments, the at least one acoustic transducer is positioned to apply a standing acoustic wave transverse to the microfluidic separation channel.

In some embodiments, the system may comprise at least two microfluidic separation channels connected in parallel and a manifold configured to distribute the pretreated biofluid to the at least two microfluidic separation channels.

In some embodiments, the bifunctional antibodies comprise at least one binding site having a non-specific affinity.

In some embodiments, the bifunctional antibodies have at least one binding site having affinity for a cluster-forming cell.

The system may further comprise a source of the cluster-forming cells in fluid communication with the source of the biofluid.

In some embodiments, the system may further comprise a control module in electrical communication with the source of the cluster-forming cells, configured to introduce a predetermined amount of the cluster-forming cells into the biofluid in response to a concentration of the cluster-forming cells and/or a concentration of the non-target cells in the biofluid.

In accordance with another aspect, there is provided a kit for microfluidic cell separation. The kit may comprise at least one microfluidic separation channel comprising at least one inlet, a first outlet, and a second outlet. The kit may comprise a source of an additive fluidly connectable to the source of the biofluid, the additive comprising a cocktail of bifunctional antibodies selected to bind the non-target cells to form non-target cell clusters. The kit may comprise at least one acoustic transducer configured to be coupled to a wall of the at least one microfluidic separation channel. The kit may comprise instructions to provide a biofluid, pretreat the biofluid by introducing a predetermined volume of the additive into the biofluid to form a pretreated biofluid comprising the target cells and the non-target cell clusters, flow the pretreated biofluid into the at least one inlet of the microfluidic separation channel, and apply acoustic energy to the microfluidic separation channel to separate the target cells from the non-target cell clusters.

In some embodiments, the bifunctional antibodies comprise at least one binding site having a non-specific affinity.

In some embodiments, the cocktail of the bifunctional antibodies is selected or designed responsive to a measured or expected cell population of the biofluid.

In some embodiments, the additive is substantially free of capture particles.

In accordance with another aspect, there is provided a method of facilitating separation of target cells from non-target cells in a biofluid. The method may comprise providing at least one microfluidic separation channel comprising at least one inlet, a first outlet, and a second outlet. The method may comprise providing a source of an additive fluidly connectable to the source of the biofluid, the additive comprising a cocktail of bifunctional antibodies selected to bind the non-target cells to form non-target cell clusters. The method may comprise providing at least one acoustic transducer configured to be coupled to a wall of the at least one microfluidic separation channel. The method may comprise providing instructions to pretreat the biofluid by introducing a predetermined volume of the additive into the biofluid to form a pretreated biofluid comprising the target cells and the non-target cell clusters, flow the pretreated biofluid into the at least one inlet of the microfluidic separation channel, and apply acoustic energy to the microfluidic separation channel to separate the target cells from the non-target cell clusters.

In some embodiments, the bifunctional antibodies comprise at least one binding site having a non-specific affinity.

In some embodiments, the cocktail of the bifunctional antibodies is selected or designed responsive to a measured or expected cell population of the biofluid.

The method may comprise providing a control module configured to introduce the predetermined volume of the additive into the biofluid to produce the pretreated biofluid.

In some embodiments, the control module is configured to direct a pump to flow the pretreated biofluid into the at least one inlet of the microfluidic separation channel and direct the acoustic transducer to apply the acoustic energy to the microfluidic separation channel.

In some embodiments, the additive is substantially free of capture particles.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic drawing of a microfluidic separation channel, according to one embodiment;

FIG. 2 is a schematic drawing of an alternate microfluidic separation channel, according to another embodiment;

FIG. 3 is a micrograph of a microfluidic separation channel coupled to an acoustic transducer that is turned off;

FIG. 4 is a micrograph of a microfluidic separation channel coupled to an acoustic transducer that is turned on;

FIG. 5 is a schematic drawing of an exemplary acoustic separation of T cells using a microfluidic separation channel, according to one embodiment;

FIG. 6 is a schematic drawing of an alternate microfluidic separation channel, according to another embodiment;

FIG. 7 is a schematic drawing of a system for microfluidic cell separation, according to one embodiment;

FIG. 8 is a schematic drawing of an alternate system for microfluidic cell separation, according to another embodiment;

FIG. 9 is a graph comparing lymphocyte to monocyte separation ratio in the fraction collected after separation, according to one embodiment of a method of separating cells in a biofluid, according to one embodiment;

FIG. 10 is a graph of the percentage of recovery for lymphocytes and other white blood cells from leukapheresis fluid, buffy coat, and whole blood after separation according to one embodiment of a method of separating lymphocytes and white blood cells from other cells in a biofluid, according to one embodiment;

FIG. 11 is a graph of lymphocyte purity as a percentage of all white blood cells from leukapheresis fluid, buffy coat, and whole blood after separation according to one embodiment of a method for separating lymphocytes from other cells in a biofluid, according to one embodiment;

FIG. 12A is a graph showing recovery of specific cell types in an output suspension obtained by varying voltage, according to one embodiment;

FIG. 12B is a graph showing recovery of specific cell types in an output suspension obtained by varying voltage, according to one embodiment;

FIG. 12C is a graph showing purity of input and output suspensions, according to one embodiment;

FIG. 12D is a graph showing recovery of specific cell types in an output suspension obtained by varying voltage, according to one embodiment;

FIG. 12E is a graph showing recovery of specific cell types in an output suspension obtained by varying voltage, according to one embodiment;

FIG. 13A is a graph showing cell count for input, output, low RBC and high RBC samples, according to one embodiment;

FIG. 13B is a graph showing cell viability after acoustic separation, according to one embodiment;

FIG. 14A is a graph showing purity of input and output suspensions, according to one embodiment;

FIG. 14B is a graph showing purity of input and output suspensions, including low RBC and high RBC count suspensions, according to one embodiment;

FIG. 15A is a graph showing percent of non-target cells depleted as a function of ratio of red blood cell (RBC) to non-target cells, according to one embodiment;

FIG. 15B is a graph showing depletion of non-target cells as a function of hematocrit, according to one embodiment;

FIG. 16 includes micrographic images showing clusters of cells at increasing cluster-forming cell to non-target cell ratios, according to several embodiments;

FIG. 17 is a graph showing percent of target cells enriched in a sample after incubation with an antibody cocktail and microfluidic acoustic separation, according to one embodiment;

FIG. 18 is a schematic drawing of the microfluidic acoustophoresis device, according to one embodiment; and

FIG. 19 is a graph showing exemplary idealized theoretical trajectories of lymphocytes flowing through the microfluidic separation channel, according to one embodiment.

### DETAILED DESCRIPTION

In the fields of cell therapy and bioprocessing emerging medical techniques may involve extraction of blood or tissue from a patient followed by purification of a particular cell type from a sample. In some applications, the particular cell type is prepared for treatment or manipulated before it is re-injected into a patient. Aspects and embodiments disclosed herein relate to separation of a desired cell type from a liquid suspension of mixed cell types. In particular, one example application is the separation of leukocytes or a subclass of leukocytes from a blood sample.

Aspects and embodiments disclosed herein may relate to methods and systems for use in processing of cells for cell therapy. Many other uses of some of the embodiments described herein could also be envisioned, in particular wherever a particular cell type is desired to be collected from a cell suspension or natural sample. Some non-limiting examples include sample preparation for analysis, such as diagnostic or environmental monitoring assays, tissue engineering and cell culture, in vitro models, cell and particle purification, and biomanufacturing systems, such as for energy applications.

Formerly cell selection for applications within bioprocessing has been performed by one or more batch centrifugations, continuous centrifugations, magnetic separation, or combinations thereof. Centrifugation is only able to separate particles by density, limiting its ability to separate leukocyte subclasses from other types of leukocytes. Addition of a density medium may improve leukocyte stratification, but only in small batch procedures requiring technically trained operators. Furthermore, no form of centrifugation is able to separate subclasses of lymphocytes such as T cells from B cells.

Magnetic separation can be highly selective but depends on the attachment of paramagnetic capture particles to cells using affinity ligands, such as antibodies. The particles may pose a safety risk if injected into a patient. Accordingly, the magnetic particles must be removed from a final therapeutic product. The efficiency of magnetic separation varies with the load of interfering cells or with the concentration of background proteins contained in the sample that may specifically or non-specifically bind to the affinity ligand. Additionally, the attachment of certain magnetic particles to cells through affinity ligands may be irreversible. In this case, only one separation can be done using a general magnetic force. For these reasons, magnetic separation is a complex procedure that is usually insufficient for bioprocessing workflow.

Aspects and embodiments disclosed herein may be advantageous over previous cell separation technologies because, for example, in some embodiments the purification of desired cells can be performed continuously, in some embodiments, the systems and methods provide separation by both size and density to further enhance cell separation, in some embodiments the separation processes may be readily scaled to small or large sample volumes, in some embodiments, a high degree of purification can be achieved without the use of capture or magnetic particles, or other foreign particles, and in some embodiments, further purification can be achieved with the addition of safely injectable, physiologically acceptable additives.

One non-limiting example of cell therapy that may be performed using systems and methods described herein is CAR-T therapy for the treatment of blood cancers. The therapy may involve engineering chimeric antigen receptors on T-cells by viral transduction or other gene editing methods known to those skilled in the art. In CAR-T treatment, blood is generally collected from a patient. The blood may be whole blood, or leukapheresis product. Leukapheresis product is a collection of mainly leukocytes and platelets, with a reduced concentration of erythrocytes, as compared to whole blood.

From this collected blood sample, specific subclasses of the leukocytes may be selected for further processing. In CAR-T therapy, the desired classes of cells may vary, but generally include mononuclear cells, lymphocytes, T cells, or subclasses of T cells, such as CD4+, or CD8+. The selected cells may then be modified (transduced) by genetic engineering to enhance their ability to attack malignant cells. The genetic engineering may include incubating to increase their abundance, washing or purifying, testing for quality control, and optionally infusing into a patient.

The aspects and embodiments disclosed herein may improve methods for selecting the desired cells and may also have applications in other steps of the process such as washing of the cells, or purification of samples after transduction.

Acoustic separation, also referred to as acoustophoresis, may be used to isolate or enrich desired cells as part of a bioprocessing workflow. Acoustic separation of particles in a biofluid has been described in, for example, U.S. Patent Application Publication No. 2020-0057045, U.S. Patent Application Publication No. 2021-0293781, U.S. Patent Application Publication No. 2019-0290829, U.S. Patent Application Publication No. 2019-0388606, U.S. Patent Application Publication No. 2016-0030660 (now U.S. Pat. No. 10,099,002), U.S. Patent Application Publication No. 2016-0008532 (now U.S. Pat. No. 10,166,323), and U.S. Patent Application Publication No. 2013-0048565 (now U.S. Pat. No. 9,731,062), and in U.S. Pat. Nos. 9,504,780, 9,974,898, 10,661,005, 10,914,723, and 11,291,756, each of which is herein incorporated by reference in their entirety.

The aspects and embodiments disclosed herein provide separation of a desired cell type, for instance a target cell, from a liquid suspension of mixed cell types including other non-target cell types. More specifically, the aspects and embodiments disclosed herein provide selective separation between cell types, without requiring the use of an affinity-based capture particle. Thus, the methods disclosed herein may be performed substantially free of capture particles, such as magnetic particles.

In acoustic separation, a mixed suspension may flow through a duct that is oscillated at ultrasonic frequencies by an external mechanical oscillator. The duct may form a resonant cavity, for instance so that ultrasonic pressure waves are generated and contact the flow across the duct. For example, the ultrasonic waves may be generated at an angle relative to the flow. Ultrasonic waves may be generated in a direction substantially transverse to the flow. Cells or other particles in the suspension may experience a force from the pressure waves and migrate to nodes in the resulting pressure field. The rate at which the cells migrate generally depends on particle size, density, and compressibility. Separation may be facilitated, for example, by larger and more dense cells migrating to a pressure node, with smaller or neutrally buoyant cells migrating slowly, not migrating (substantially staying on axis), or migrating to anti-nodes. For instance, in a typical configuration separation process, the pressure node is established along the axis of the duct and certain particles may move to this pressure node axis and flow in a concentrated stream along it, while other cells may remain disperse or move to a pressure anti-node axis.

Referring again to the example application of CAR-T therapy, lymphocytes may be preferentially extracted from blood samples. The therapy may involve, for example, forming clusters of undesired cells, such as erythrocytes and other classes of leukocytes. The lymphocytes may be less susceptible to acoustic energy than the clusters of undesired cells. Therefore, when a cell suspension, for example a blood sample, is passed through an acoustic separator, lymphocytes may remain in a side stream with greater abundance than clusters of undesired cells. The side stream may be collected for processing and the center stream may be discarded. In certain embodiments, the methods may additionally comprise altering a property of the cell suspension or of a certain class of cells within the suspension to further enhance the separation.

In accordance with an aspect, there is provided a method of separating target cells from non-target cells in a biofluid. More specifically, there is provided a method for selective, differential separation of a desired cell type from a biofluid comprising a suspension of mixed cell types. Target cells which may be selectively separated from the mixed cell types in the suspension include leukocytes, mononuclear cells, lymphocytes, monocytes, granulocytes, agranulocytes, macrophages, T cells, B cells, NK cells, subclasses thereof, and combinations thereof. For instance, in some embodiments, target cells are subclasses of T cells, including but not limited to CD4+, CD8+, T_{H}, T_{CM}, and T_{FH} cells. In some embodiments, target cells are selected to be stem cells.

Non-target cells may comprise any and all cells not selected as the target cell. Non-target cells may comprise erythrocytes, platelets, granulocytes, monocytes, macrophages, leukemic cells, and leukocytes, excluding any leukocytes selected as target cells. In some embodiments, the non-target cells are platelets and erythrocytes. Erythrocytes are approximately the same size as lymphocytes. In order to separate erythrocytes from lymphocytes in a biofluid, efficiency may be greatly increased by forming clusters of erythrocytes and/or other non-target cells.

In certain exemplary embodiments, the methods may include introducing an additive to alter or regulate at least one parameter of the biofluid, target cells, or non-target cells. For instance, the additive may alter the aggregation potential of non-target cells and/or the density of the biofluid. According to certain embodiments, the additive is introduced in sufficient volume to regulate the density of the biofluid to be substantially similar to the density of the lymphocytes. However, it has been surprisingly discovered that efficient separation of lymphocytes from non-target cell clusters may be obtained without the use of an additive that alters or regulates a parameter of the biofluid, target cells, or non-target cells.

According to certain embodiments, target cells are separated from non-target cells to produce a target cell enriched fluid. The target cells and/or non-target cells may be live cells, frozen cells, preserved cells, or cells grown in a cell culture. The target cell enriched fluid may comprise a higher concentration of target cells, as compared to the input biofluid or the pretreated biofluid.

Generally, a biofluid, for example whole blood, comprises a high concentration of erythrocytes. To produce a target cell enriched fluid, it may be desirable to selectively deplete erythrocytes.

The method of separating target cells from non-target cells in a biofluid may further comprise providing a biofluid. In some embodiments, the biofluid may be obtained from a donor subject. The donor subject's biofluid may be subjected to down-stream processes directly, or may be collected and stored for later processing. As used herein, “directly” refers to processing of the biofluid without subjecting the biofluid to a long-term storage period. For instance, the biofluid may be processed immediately in an in-line arrangement, within minutes, or within hours. The biofluid may be stored for one day or more. In some embodiments, the biofluid is collected from a donor subject through an intraluminal line. Accordingly, the method may further comprise obtaining the biofluid from a donor subject through an intraluminal line. As used herein, an “intraluminal” line refers to a transfusion line connectable to a lumen of a subject. More specifically, an intraluminal line may be connectable to a body cavity, tubular structure, or organ in the body, such as a vein, an artery, the bladder, or intestine. For instance, a transfusion line may be connectable to the circulatory or gastrointestinal system of the subject. The intraluminal line includes, for example, intravenous lines, central venous lines, intravascular lines, intratissue lines, catheters, and transfusion lines. The intraluminal line catheter may be, for example, a peripheral indwelling catheter, an intravenous catheter, or a central venous catheter.

As used herein, the term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

In accordance with certain embodiments, the biofluid may be obtained from a standard blood processing device. For instance, the biofluid may be obtained from an apharesis machine. The biofluid may be directly obtained from a standard blood processing device and further processed immediately, for example in an in-line arrangement. In other embodiments, the biofluid may be obtained from a standard blood processing device and stored for one day or more before being introduced into the microfluidic separation chamber.

In some embodiments, the method further comprises selecting the biofluid from blood buffy coat, leukapheresis product, peripheral blood, whole blood, lymph fluid, synovial fluid, spinal fluid, bone marrow, ascities fluid, and combinations or subcomponents thereof. The biofluid may comprise a synthetic medium comprising a cell suspension. For instance, the biofluid may comprise a cell culture medium. In some embodiments, the biofluid may comprise a subcomponent of a biofluid. For instance, the biofluid may comprise cell enriched biofluid, cell depleted biofluid, diluted biofluid, concentrated biofluid, filtered biofluid, purified biofluid, or otherwise treated biofluid.

As used herein, leukapheresis product refers to a blood product which has undergone an apheresis separation process. The apheresis separation process may have been performed to deplete or enrich for leukocytes. Thus, the leukapheresis product may comprise leukocyte enriched apheresis product or leukocyte depleted apheresis product. In some embodiments, the leukapheresis product may comprise synthetic biofluid. In some embodiments, the leukapheresis product may be purchased from a manufacturer. In some non-limiting embodiments, the leukapheresis product is LeukoPak™ leukapheresis product, as distributed by AllCells (Alameda, Calif.).

The method of separating target cells from non-target cells in a biofluid may further comprise pretreating the biofluid. In some embodiments, pretreating the biofluid comprises introducing an additive into the biofluid. The additive may be cell friendly. For instance, in some embodiments, the concentration of additive introduced into the biofluid is generally safe for intraluminal injection into a subject. In some embodiments, the additive selected is physiologically acceptable and generally safe for intraluminal injection into a subject.

In certain embodiments, the additive may be a cocktail of antibodies selected to bind cells. The additive may comprise the cocktail of antibodies in a carrier. The carrier may be or comprise a liquid carrier, such as water, deionized water, saline solution (e.g., phosphate buffered saline (PBS)), cell media, a density gradient medium, or a combination thereof. In some embodiments, the additive may be substantially free of capture particles. For example, the additive may be substantially free of magnetic capture particles and particles designed to have a selected acoustic buoyancy. Thus, in some embodiments, pretreating the biofluid comprises introducing into the biofluid the cocktail of bifunctional antibodies without a capture particle.

In general, the additive may comprise unbound antibodies. The unbound antibodies may have two or more binding sites, for example, 2-20 binding sites, 2-10 binding sites, 2-6 binding sites, 6-10 binding sites, or 10-20 binding sites. The antibodies may be bifunctional or multifunctional antibodies. As disclosed herein, bifunctional antibodies may be configured to bind two distinct cell types or moieties in the biofluid, having at least one binding site for each cell type or moiety. Multifunctional antibodies may be configured to bind more than two distinct cell types or moieties, having at least one binding site for each cell type or moiety.

In certain exemplary embodiments, the antibodies may target specific surface markers, such as glycoproteins, on target or non-target cells, allowing for targeted separation. The surface markers may be specific enough to target subclasses of lymphocytes. Clusters or “rosettes” of cells may be formed with tetrameric antibody cocktails that bind multiple types of cells.

In some embodiments, the unbound antibodies may be selected, designed, or engineered to bind non-target cells and have at least one non-specific binding site. For instance, bifunctional antibodies may have at least one binding site selected for a class of non-target cells and at least one binding site having non-specific affinity. Multifunctional antibodies may have at least one binding site selected for more than one class of non-target cells and at least one binding site having non-specific affinity.

In certain embodiments, the additive may be a cocktail of antibodies selected to bind the non-target cells, leaving target cells unbound. The antibodies may selectively bind the one or more class of non-target cells forming non-target cell clusters. In certain embodiments, the antibodies may bind the non-target cells to cluster-forming cells forming the non-target cell clusters. In other embodiments, the antibodies may bind non-target cells to one another forming the non-target cell clusters. Thus, in some embodiments, the methods may be performed substantially free of cluster-forming cells.

The method may comprise selecting, designing, or engineering the cocktail of antibodies, for example, responsive to a known or expected cell population (for example, concentration of one or more cell type) of the biofluid. Thus, in some embodiments, the methods may comprise determining a cell population (for example, measuring concentration of one or more cell type) of the biofluid.

In some embodiments, the antibodies may be selected to bind all or some non-target cells to form the non-target cell clusters. In some embodiments, the antibodies may be selected to bind all or some non-target cells to cluster-forming cells to form the non-target cell clusters.

The cluster-forming cells may comprise a class of non-target cells. In certain exemplary embodiments, the cluster-forming cells may comprise erythrocytes. In certain exemplary embodiments, the cluster-forming cells may comprise platelets. The cluster-forming cells may comprise leukocytes selected from the group consisting of mononuclear cells, lymphocytes, monocytes, granulocytes, agranulocytes, macrophages, T cells, B cells, NK cells, subclasses thereof, and combinations thereof.

In certain embodiments, the additive may be a cocktail of antibodies selected to bind the target cells, leaving all or some non-target cells unbound. The antibodies may bind the target cells forming target cell clusters. The method may comprise selecting the cocktail of antibodies to bind target cells. In some embodiments, the antibodies may be selected to bind target cells to cluster-forming cells to form the target cell clusters. Thus, any of the embodiments disclosed herein may be employed to bind and form target cell clusters.

In certain exemplary embodiments, the methods disclosed herein may be employed to separate a class or subclass of leukocytes, for example, a class of lymphocytes. In particular, the antibodies may be designed to selectively bind all or some non-target classes or subclasses of leukocytes, leaving a selected target class of leukocytes unbound. Optionally, the antibodies may be selected, designed, or engineered to selectively bind all or some non-target classes or subclasses of leukocytes to erythrocytes or platelets. Thus, the cocktail of antibodies may be specifically designed for a target application, increasing efficiency of separation.

The antibodies may be selected, designed, or engineered to selectively bind one or more class of leukocytes selected from the group consisting of mononuclear cells, lymphocytes, monocytes, granulocytes, agranulocytes, macrophages, T cells, B cells, NK cells, and subclasses thereof, optionally to cluster-forming cells. The antibodies may be selected, designed, or engineered to selectively bind one or more class of T cells, B cells, M cells, and/or NK cells, such as, CD3+ T cells, CD19+ B cells, CD14+ M cells, and CD56+ NK cells, optionally to cluster-forming cells. The antibodies may be selected, designed, or engineered to selectively bind one or more class of T cells, such as CD4+, CD8+, T_{H}, T_{CM}, and T_{FH} cells, optionally to cluster-forming cells. The exemplary cells and classes of cells described herein may be target cells or non-target cells.

In certain exemplary embodiments, the cocktail of antibodies may be selected to bind B cells, NK cells, and/or monocytes, optionally to cluster-forming cells, such as erythrocytes or platelets, leaving T cells unbound. In other exemplary embodiments, the cocktail of antibodies may be selected to bind T cells, NK cells, and/or monocytes, optionally to cluster-forming cells, such as erythrocytes or platelets, leaving B cells unbound. In other exemplary embodiments, the cocktail of antibodies may be selected to bind B cells, T cells, and/or monocytes, optionally to cluster-forming cells, such as erythrocytes or platelets, leaving NK cells unbound. The cocktail of antibodies may be selected, designed, or engineered for any target cell/non-target cell combination.

In accordance with certain embodiments, the methods may comprise controlling the ratio of the cluster-forming cells to non-target cells in the biofluid. It has been surprisingly found that improved separation may be achieved with the cocktail of antibodies by controlling the ratio of cluster-forming cells to non-target cells in the biofluid. Thus, in some embodiments, the method may comprise measuring one or both of concentration of cluster-forming cells and concentration of non-target cells. However, in other embodiments, the concentration of cluster-forming cells and/or concentration of non-target cells may be estimated based on known properties of the biofluid.

The ratio of cluster-forming cells to non-target cells may be controlled by introducing a source of the cluster-forming cells into the biofluid and/or selectively removing cluster-forming cells from the biofluid before introducing the cocktail of antibodies. In some embodiments, the biofluid may comprise a concentration of cluster-forming cells. Thus, the method may comprise introducing at least some of the cluster-forming cells into the biofluid to increase the concentration and/or removing at least some of the cluster-forming cells from the biofluid to decrease the concentration. In some embodiments, at least some of the cluster-forming cells in the biofluid may be removed by microfluidic acoustic separation, in accordance with the methods disclosed herein. Thus, at least in some embodiments, a cluster-forming cell depleted output suspension may be combined with an additive comprising a cocktail of antibodies and introduced into a microfluidic acoustic separation device.

In certain exemplary embodiments, introducing a source of the cluster-forming cells into the biofluid to increase a ratio of cluster-forming cells to non-target cells, wherein the cluster-forming cells comprise erythrocytes or platelets, may comprise introducing a predetermined volume of whole blood into the biofluid. In some embodiments, the whole blood may be extracted from a donor subject. Thus, in some embodiments, the method may comprise obtaining the source of the cluster-forming cells from the donor subject. In some exemplary embodiments, the method may comprise obtaining a biofluid from the subject, separating the biofluid into a source of cluster-forming cells and a biofluid to be processed, and introducing a controlled amount of the cluster-forming cells into the biofluid to control the ratio of cluster-forming cells to non-target cells in the biofluid.

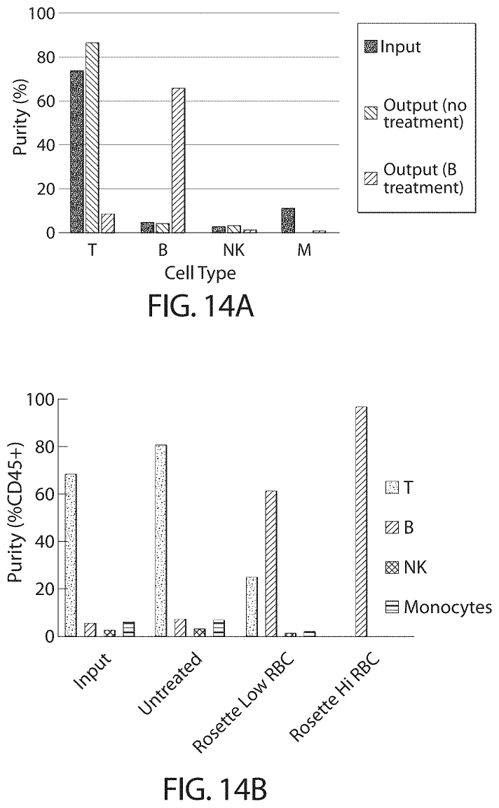

The predetermined amount of the cocktail of antibodies or predetermined volume of the additive may be selected responsive to the concentration of cluster-forming cells and/or the ratio of cluster-forming cells to non-target cells. FIG. 14 is a graph showing percent depleted non-target cells for varying a ratio of cluster-forming cells (erythrocytes in this example) to non-target cells in the biofluid. As shown in FIG. 14, the ratio of cluster-forming cells to non-target cells may be selected to be between about 10:1 and 100:1, for example, between about 10:1 and 60:1 or between about 10:1 and 25:1. The ratio of cluster-forming cells to non-target cells may be greater than 10:1, greater than 20:1, greater than 30:1, greater than 40:1, greater than 50:1, greater than 60:1, greater than 70:1, or greater than 80:1. The ratio of cluster-forming cells to non-target cells may be less than 60:1, less than 50:1, less than 40:1, less than 30:1, less than 20:1, or less than 15:1. The ratio of cluster-forming cells to non-target cells may be optimized to achieve improved purity of target cells in the output suspension. It is hypothesized that higher ratios, however, may interfere with the purification and therefore there is expected to be an optimum ratio or range of ratios for acoustophoretic separation.

In certain exemplary embodiments, improved separation was achieved with a smaller ratio of cluster-forming cells to non-target cells. For instance, excellent separation was achieved with less than 20:1 erythrocytes to non-target leukocytes. The results were surprising and significant. In particular, the results indicate that the separation methods may be effectively performed with whole blood samples. For instance, in certain embodiments, the biofluid may be whole blood. Thus, in certain embodiments, a first separation to obtain leukapheresis product from a whole blood sample may not be necessary.

One exemplary cocktail of antibodies is included in the RosetteSep™ or EasySep™ cell enrichment cocktail kits, (distributed by StemCell Technologies, Vancouver, Calif.). RosetteSep™ and EasySep™ cell enrichment cocktail kits typically include (or are designed for use with) one or more antibody complexes having affinity for a magnetic bead, magnetic beads, and a density gradient media or other density gradient media (such as a RosetteSep™ density gradient media). RosetteSep™ and EasySep™ kits are typically used for separation of cells in a centrifugation process. However, separation by centrifugation is not the same as separation by acoustophoresis. For instance, an antibody that binds B cells to other B cells is not expected to be effective at purifying T cells by centrifugation, but is expected to be effective at purifying T cells by acoustophoresis. Furthermore, RosetteSep™ and EasySep™ kits may not be designed or optimized for acoustophoretic separation of a specific target cell of interest. Thus, for certain target cells, the cocktail of antibodies may be selected, designed, or engineered to selectively bind the non-target cells and leave target cells unbound, increasing efficiency of separation in an acoustic microfluidic setting.

Additionally, the cocktail of antibodies that bind non-target cells (optionally to cluster-forming cells) may be selected based on known or expected non-target cells of interest. In particular, a general cocktail of antibodies, such as those included in RosetteSep™ cocktail kits, may include antibodies targeting moieties that are not of interest, such as moieties not expected to be present in the biofluid sample and/or moieties that are already capable of separation by acoustophoresis, which need not be formed in a cluster for effective separation. Thus, the method may include selecting or designing the cocktail of antibodies to bind only those non-target cells which are of interest, optionally to cluster-forming cells, leaving other cell types unbound.

In some embodiments, the non-target cells of interest may be the non-target cells known or expected to be present in the mixed cell sample. In some embodiments, the non-target cells of interest may be those non-target cells which are otherwise difficult to separate by acoustophoresis (for example, due to having a similar size and/or other similar properties as the target cells), optionally leaving other non-target cells in the sample unbound. Selecting or designing the antibodies to bind only with those non-target cells of interest, optionally to cluster-forming cells, may increase efficiency of separation in an acoustic microfluidic setting.

In one exemplary embodiment, an off-the-shelf antibody cocktail may include antibodies that bind monocytes to erythrocytes. However, monocytes may be effectively separated from T cells and B cells by acoustophoresis without alteration. Thus, the methods may comprise employing an antibody cocktail designed to bind non-target cells and leave monocytes unbound. In such exemplary embodiments, the monocytes are non-target cells that are not part of the group of non-target cells of interest.

The methods may include introducing into the biofluid a predetermined amount of the cocktail of antibodies or predetermined volume of the additive. The predetermined amount or volume may be selected based on a property of the biofluid. For instance, the predetermined amount or volume may be selected based on flow rate, concentration of target cells, concentration of non-target cells, concentration of cluster-forming cell, density, hematocrit (HCT %), or another property of the biofluid. Thus, the volume of the additive may be controlled to increase efficiency of separation. In some embodiments, the methods may comprise measuring or determining the property of the biofluid and selecting the predetermined amount of the cocktail of antibodies or predetermined volume of the additive responsive to the measured or determined property. In other embodiments, the property may be estimated based on known or expected properties of the biofluid. Thus, the methods may comprise selecting the predetermined amount of the cocktail of antibodies or predetermined volume of the additive responsive to an estimate, known, or expected property of the biofluid.

In accordance with certain embodiments, the method may further comprise introducing an additive to modify the biofluid or cell chemistry, to further enhance separation of target cells from non-target cells. In some embodiments, pretreating the biofluid comprises introducing an additive into the biofluid to alter at least one of size of the target cells, size of the non-target cells, compressibility of the biofluid, compressibility of the target cells, compressibility of the non-target cells, aggregation potential of the target cells, aggregation potential of the non-target cells, density of the biofluid, density of the target cells, density of the non-target cells, or any combination thereof.

Exemplary additives that may alter aggregation potential include cell aggregators, such as long chain polysaccharides, cell activators, such as platelet activators or cell adhesion molecules (CAM), and antibodies or antibody fragments. The CAM may be released or obtainable from an activated platelet granule. Exemplary additives that may alter density include density gradient media. Density gradient media is a media for cell isolation, generally used in the practice of centrifugal separation. Density gradient media are well known in the art and include, for example, ACCUSPIN™ media, Histodenz™ media, OptiPrep™ media, and Histopaque® media distributed by Sigma-Aldrich (St. Louis, Mo.), Ficoll-Paque™ media and Percoll™ media distributed by GE Healthcare (Chicago, Ill.), RosetteSep™ density gradient media and Lymphoprep™ density gradient media distributed by STEMCELL Technologies (Vancouver, Canada).

The method of separating target cells from non-target cells may further comprise flowing biofluid into an inlet of a microfluidic separation channel. For instance, the method may comprise flowing the pretreated biofluid into the microfluidic separation channel. The biofluid may have a flow rate of between about 0.03 mL/min to about 0.5 mL/min. In some embodiments, the biofluid may have a flow rate through the microfluidic separation channel of between about 0.05 mL/min to about 0.5 mL/min, about 0.1 mL/min to about 0.5 mL/min, about 0.1 mL/min to about 0.4 mL/min, or about 0.1 mL/min to about 0.3 mL/min. The biofluid may have a flow rate through the microfluidic separation channel of about 0.03 mL/min, 0.05 mL/min, 0.08 mL/min, 0.1 mL/min, 0.2 mL/min, 0.3 mL/min, 0.4 mL/min, 0.5 mL/min, or any range therebetween.

The method may further comprise applying acoustic energy to the microfluidic separation channel. In some embodiments, the acoustic energy is applied in the form of an acoustic wave. The acoustic wave may be applied at an angle relative to the flow of fluid through the separation channel. The angle and magnitude of the acoustic wave may be engineered based on size of the device, size of the channel, or flow rate of fluid through the channel.

In some embodiments, the acoustic energy may be applied in a direction substantially transverse to the biofluid flow through the microfluidic separation channel. The acoustic wave may be a standing acoustic wave. In some embodiments, the acoustic energy may be applied to the microfluidic separation channel continuously. The continuous application of acoustic energy may allow for a greater efficiency of separation. In alternate embodiments, the acoustic energy may be applied to the microfluidic separation channel intermittently or on a timed schedule. The intermittent energy application may allow for cells to move freely through the channel if there is a blockage.

Applied acoustic energy may generally act on the cells and particles within the biofluid to drive them according to size, density, and/or compressibility. As disclosed herein, the cell clusters may respond to the applied acoustic energy as a larger sized particle. Thus, cell clusters may be separated from unbound cells by the applied acoustic energy in accordance with a size differential of the cell cluster as compared to the unbound cell.

In some embodiments, the method may comprise accumulating target cells within a primary stream along the separation channel. In some embodiments, the method may comprise accumulating non-target cells within a secondary stream along the separation channel. In some embodiments, the target cells may form the cell cluster while non-target cells are unbound cells. In some embodiments, the target cells may be unbound cells, while the non-target cells form the cell cluster. The accumulation of a cell type within a desired stream along the separation channel may be engineered by adjusting parameters such as wavelength, frequency, amplitude, power level, or other modulation of the applied acoustic energy.

Depending on the target cells or non-target cells selected according to the method and whether the target cells or non-target cells are formed in the cell cluster, one class of cells may accumulate in response to a pressure node or anti-node generated by the acoustic energy. For instance, target cells may accumulate within a primary stream in response to a pressure node, and non-target cells may accumulate within a secondary stream in response to a pressure anti-node. Generally, particles, including cells, will be driven by the acoustic energy in response to their contrast factor. Particles may migrate at a rate which is proportional to the magnitude and sign of their contrast factors. In some embodiments, particles with a positive contrast factor are driven to pressure nodes, while particles with a negative contrast factor are driven to pressure anti-nodes. Particles with a greater magnitude contrast factor are generally driven at a faster rate than particles with a lesser magnitude contrast factor.

The rate at which cells are driven in response to their acoustic energy generally depends on particle size, density, and compressibility. Briefly, the contrast factor is based on the bulk modulus (K) and density (p) of a particle, here of the cells or cell clusters. When suspended in a fluid, the contrast factor ((p) for the cells or clusters is calculated with the below equation:

$$φ = \frac{5 ρ - 2 \cdot 1.02}{2 ρ + 1.02} + \frac{2.2}{K}$$

In certain embodiments, the acoustic energy may act on non-target cells in the form of non-target cell clusters. Any separation of non-target cells disclosed herein may include non-target cell clusters. The non-target cell clusters may respond to the acoustic energy as does a particle having a greater size. Thus, the rate at which non-target cells in clusters are driven in response to their acoustic energy is generally greater than the non-target cells alone.

In some embodiments, the method of separating target cells from non-target cells in a biofluid comprises collecting the at least one primary stream comprising the target cells. Generally, the biofluid entering the microfluidic separation channel is a well-mixed primary stream, comprising desegregated target cells and non-target cells. Upon experiencing acoustic energy, target cells and non-target cells may generally accumulate into fractions of the general stream of biofluid. The fraction or fractions of biofluid flowing through the microfluidic separation channel selectively enriched in target cells are defined as the primary stream. There may be more than one fraction of biofluid within the microfluidic separation channel enriched in target cells. For instance, target cells may be driven to a pressure node at the center of the channel in one embodiment, and target cells may be driven to the pressure anti-nodes at the periphery of the channel in an alternate embodiment. The location of pressure nodes and anti-nodes within the channel may be designed by positioning the acoustic energy or by selecting frequency and wavelength of the acoustic waves. The primary stream comprising target cells may be collected for storage, immediate use, transfusion into a patient, or for research. In certain embodiments, where the method is designed to create a target cell depleted fluid, the primary stream comprising target cells may be discarded as waste.

Similarly, in some embodiments, the method of separating target cells from non-target cells comprises collecting the at least one secondary stream comprising non-target cells, optionally in the form of non-target cell clusters. The fraction or fractions within the biofluid selectively depleted in target cells, and selectively enriched in non-target cells are defined as the secondary stream. In certain embodiments, the target cells and non-target cells have opposing contrast factors. With opposing contrast factors, the target cells and non-target cells may be driven in opposite directions, or one may be driven away from the general stream, for example to the center or the periphery of the channel. In other embodiments, the target cells and non-target cells have contrast factors of a different magnitude, but the same sign. In these embodiments, one class of cells may be driven away at a faster rate than the other, defining the primary and secondary streams. The secondary stream may be collected for storage, for further research, or to be discarded as waste. Where the method is designed to deplete a biofluid of the target cells, the secondary stream may be collected for later use or for transfusion into a patient. The method may comprise collecting the primary stream comprising target cells and further comprise separately collecting the at least one secondary stream comprising the non-target cells.

According to certain embodiments, a target cell enriched or target cell depleted fluid may be post-treated and delivered to a recipient subject. For instance, the primary stream may be post-treated and delivered to a recipient subject. Post-treating a fluid may comprise a process such as washing, separating, concentrating, diluting, heating, purifying, or filtering capable of removing toxins, contaminants, or harmful chemical compounds from the fluid. In general, a fluid is post-treated to produce a physiologically acceptable fluid that may be directly delivered to a recipient subject, for example via an intraluminal line as previously described. The post-treated fluid may be stored for delivery to a recipient subject at a later time.

In some embodiments, the target cell enriched or target cell depleted fluid is post-treated to produce a therapeutic fluid. Post-treating the fluid may comprise viral transduction, gene transfer, or gene editing of the target cells to produce a therapeutic, physiologically acceptable fluid for delivery to a recipient subject, as previously described.

In some embodiments, the recipient subject is the same as the donor subject. In other embodiments, the donor subject and the recipient subject are different from one another. The donor subject and the recipient subject may generally be physiologically compatible.

The method may be performed in line such that the biofluid is collected from a subject and directly pretreated, target cells are separated from non-target cells in the biofluid by a method as described herein to produce a target cell enriched fluid, the fluid enriched in target cells may be post-treated, and the post-treated fluid may be directly delivered back to the subject. In some embodiments, the method is performed essentially as previously discussed, however the target cells are separated from non-target cells to produce a target cell depleted fluid, which may be post-treated and delivered back to the subject.

According to certain embodiments, the method further comprises flowing a second fluid adjacent to the biofluid into an inlet of the microfluidic separation channel. The inlet may be an inlet separate from the biofluid inlet of the microfluidic separation channel. The biofluid and the second fluid may flow through the separation channel in substantially parallel form. For instance, both fluids may flow through the separation channel at opposite peripheries of the channel, the second fluid may flow through both peripheries of the channel, or the second fluid may flow in the center of the channel. The biofluid and the second fluid may flow through the separation channel in substantially laminar form. As used herein, substantially laminar flow includes substantially ordered flow Laminar flow may have a Reynolds number (Re) less than about 2100. In certain embodiments, laminar flow has a Reynolds number (Re) less than about 4000.

In certain embodiments, the second fluid is an inert fluid that may comprise water, deionized water, or phosphate buffered saline (PBS). The second fluid may have its density adjusted with a density gradient medium or density additive, independently from the pretreated biofluid. The applied acoustic energy may drive target or non-target cells from the biofluid into the essentially parallel flowing second fluid initially comprising no cells, such that the second fluid, now comprising selectively separated cells, may exit the microfluidic separation channel through a separate outlet. Where the target cells are driven into the second fluid, the second fluid comprising target cells is essentially the primary stream. Conversely, where the non-target cells are driven into the second fluid, the second fluid is essentially the secondary stream.

According to certain embodiments, the methods described herein may be performed in a staged separation or in series. Specifically, a target cell enriched fluid or a target cell depleted fluid may be further processed by pretreating with an additive, flowing through a second microfluidic separation channel, and applying acoustic energy. The additive introduced into the fluid in the downstream operation may be the same or a different additive as the one introduced into the biofluid in the first pass separation process. Additionally, the target cells selected in the first pass process may be the same or different as those selected in the second pass process.

As a non-limiting example, a biofluid may be pretreated and flowed through a microfluidic separation channel to produce a platelet depleted fluid. The output platelet depleted fluid may further be flowed through a second microfluidic separation channel to remove neutrophils and/or monocytes. As another non-limiting example, a biofluid may be flowed through a microfluidic separation channel to produce lymphocyte enriched fluid. The lymphocyte enriched fluid may be flowed through a second microfluidic separation channel to produce a further lymphocyte enriched fluid.

In some embodiments, the first pass target cell enriched or target cell depleted fluid is recycled and reintroduced into the biofluid or into the pretreated biofluid to flow through the microfluidic separation channel as a blend.

In some embodiments, the first pass target cell enriched or target cell depleted fluid has a recovery and/or purity sufficient for the desired application. For instance, in accordance with certain methods disclosed herein, recovery and/or purity of the output suspension may be sufficient for the desired application such that a recycle or second pass of the suspension is not necessary. In some embodiments, the output suspension may have a target cell purity of greater than 90%, for example, greater than 92%, greater than 94%, greater than 96%, or greater than 98%, greater than 99%. In particular embodiments the target cell purity may be determined after a first pass separation. In some embodiments, the output suspension may have a target cell or non-target cell recovery of greater than 90%, for example, greater than 92%, greater than 94%, greater than 96%, or greater than 98%, greater than 99%. In particular embodiments the recovery rate may be determined after a first pass separation.

Separation efficiency may be reported as separation ratio, a quantitative measurement of the ratio of cells in the product. The separation Ratio for any subpopulation x, where “side” is the primary stream and “center” is the secondary stream is defined by the following formula:

$${SR}_{x} = \frac{{n}_{x , side}}{{n}_{x , side} + {n}_{x , center}}$$

The methods disclosed herein may produce a separation ratio of at least 0.9, for example, at least 0.91, 0.92, 0.93, 0.94, 0.95, 0.96, 0.97, 0.98, 0.99, 0.999, or 0.9999. In some embodiments, a first pass separation ratio may be at least 0.9, for example, at least 0.91, 0.92, 0.93, 0.94, 0.95, 0.96, 0.97, 0.98, 0.99, 0.999, or 0.9999. In some embodiments, a second pass separation ratio may be at least 0.9, for example, at least 0.91, 0.92, 0.93, 0.94, 0.95, 0.96, 0.97, 0.98, 0.99, 0.999, or 0.9999.

The fraction of the stream out the side channel (primary stream) may also be referred to as the flow split. Flow split may be defined by the following formula:

$${FR}_{side} = \frac{{V}_{side}}{{V}_{center}}$$

According to certain embodiments, the method further comprises dosing the at least one primary stream with a reagent to produce a dosed suspension. The at least one primary stream may be a target cell enriched fluid. The reagent may be selected from an antigen or activation reagent configured to biochemically induce cell activation. The biochemically induced activation may allow for selection of subclasses of types of cells, for instance lymphocytes or T cells, by exploiting the morphological changes of activated cells. In some instances, activated cells may be larger than non-activated cells and cell size may vary throughout the cell cycle. The difference in size may allow for differential separation with acoustic energy.

The method may further comprise flowing the target cell enriched fluid through a second microfluidic separation channel or through microfluidic separation channels arranged in series and applying acoustic energy to each separation channel. The dosed suspension may allow for selection of target cells at a certain stage of the cell cycle.

For instance, in some embodiments of the method, the target cells in the primary stream (after a first pass separation) may be lymphocytes. The method may further comprise separating activated lymphocytes from non-activated lymphocytes in the primary stream. The method may further comprise dosing the lymphocyte enriched fluid with a reagent to produce the dosed suspension, flowing the dosed suspension into an inlet of a second microfluidic separation channel, and applying acoustic energy to the second microfluidic separation channel Activated lymphocytes may accumulate within at least one primary stream along the second separation channel and non-activated lymphocytes may accumulate within at least one secondary stream along the second separation channel.

In some embodiments, the method may comprise measuring at least one property of the biofluid or pretreated biofluid. The method may comprise measuring an input biofluid load on the system. The method may comprise measuring at least one of flow rate, concentration of target cells, concentration of non-target cells, concentration of cluster-forming cells, density, hematocrit (HCT %), or another property of the biofluid. The method may comprise selecting the predetermined volume of the additive responsive to the measurement. The method may comprise selecting flow rate of the biofluid, flow rate of the additive, and/or acoustic energy (for example, power, voltage, and/or frequency) responsive to the measurement.

In some embodiments, the method may comprise measuring at least one property of an output suspension, for example, a primary stream or secondary stream of the output fluid. The method may comprise measuring at least one of flow rate, concentration of target cells, concentration of non-target cells, concentration of non-target cell clusters, density, hematocrit (HCT %), or another property of an output suspension. The method may comprise selecting the predetermined volume of the additive responsive to the measurement. The method may comprise selecting flow rate of the biofluid, flow rate of the additive, and/or acoustic energy (for example, power, voltage, and/or frequency) responsive to the measurement.

In some embodiments, the method may comprise measuring at least one parameter within the microfluidic separation channel, for example, pressure, temperature, or acoustic energy within the microfluidic separation channel. The method may comprise selecting the predetermined volume of the additive responsive to the measurement. The method may comprise selecting flow rate of the biofluid, flow rate of the additive, and/or acoustic energy (for example, power, voltage, and/or frequency) responsive to the measurement.

In accordance with another aspect, there is provided a system for microfluidic cell separation. The system may be configured to separate target cells from non-target cells in a biofluid. In some embodiments, the system comprises at least one microfluidic separation channel comprising at least one inlet and at least one outlet. The at least one outlet may be a branched outlet, branching in a direction substantially away from the separation channel. In some embodiments, the microfluidic separation channel comprises a first outlet and a second outlet. The at least one inlet may be configured to receive biofluid and the at least one outlet may be configured to discharge the biofluid that has been subjected to acoustic energy. As the fluid flows through the microfluidic separation channel, it may be subjected to acoustic energy that drives the target cells and/or non-target cells towards pressure nodes and anti-nodes within the channel. In some embodiments, the first outlet is configured to discharge target cell enriched fluid and the second outlet is configured to discharge target cell depleted fluid.

The microfluidic separation channel may be formed of rigid materials. The rigid materials may have a high acoustic contrast with the biofluid. In alternate embodiments, the microfluidic separation channel may be formed of relatively elastic materials. The more elastic materials may have a lower acoustic contrast with the biofluid. However, they may form good acoustic resonators that provide low acoustic impedance and provide relatively little wave energy loss in wave transfer. The materials to form the microfluidic separation channel may include silicon, glass, metals, thermoplastics, and combinations thereof.

In some embodiments, the microfluidic separation channel may be formed of a thermoplastic material. The thermoplastic microfluidic separation channel may be small, disposable, relatively safer to handle than, for example, the glass or metal separation channels, and relatively less expensive to manufacture than the silicon, glass, or metal separation channels. In some embodiments, the thermoplastic microfluidic separation channels are manufactured by injection molding. The thermoplastic material may comprise polystyrene, acrylic (polymethyl methacrylate), polysulfone, polycarbonate, polyethylene, polypropylene, cyclic olefin copolymer, silicone, liquid crystal polymer, polyvinylidene fluoride, and combinations thereof. The microfluidic separation channel may be disposable.

In some embodiments, the microfluidic separation channel has a channel width of between about 0.2 mm to about 0.8 mm. The microfluidic separation channel may be about 0.2 mm, about 0.3 mm, about 0.4 mm, about 0.5 mm, about 0.6 mm, about 0.7 mm, or about 0.8 mm wide. In some embodiments, the microfluidic separation channel is between about 15 mm and about 35 mm long. The microfluidic separation channel may be about 15 mm, about 20 mm, about 25 mm, about 30 mm, or about 35 mm long. The microfluidic separation channel width may be correlated to the acoustic wave wavelength, such that each channel contains a pressure-node and/or pressure anti-node generated by the acoustic energy.

The system may further comprise a source of biofluid in fluid communication with the microfluidic separation channel. The source of the biofluid may be a vessel or chamber in fluid communication with the at least one inlet of the microfluidic separation channel, configured to deliver biofluid to the separation channel. The source of the biofluid may be a mixing chamber configured to receive an additive or a second fluid to be introduced into the biofluid prior to flowing the biofluid through the microfluidic separation channel. The source of the biofluid may be heated, cooled, or mixed.

In some embodiments, the source of the biofluid is fluidly connected downstream of an intraluminal line and configured to receive biofluid directly from a donor subject. The source of the biofluid may further be fluidly connected downstream to a biofluid sample, for instance a sample collected in a bag, vessel, tank, or other chamber.

In some embodiments, the system comprises a source of additive in fluid communication with the source of the biofluid, configured to introduce at least one additive into the biofluid. The additive may comprise a cocktail of antibodies selected to bind non-target cells to cluster-forming cells. In some embodiments, the additive may be substantially free of capture particles. The source of the additive may be a chamber, vessel, or tank comprising the additive. In some embodiments, the source of the additive may be heated, cooled, or comprise a mixer.

In some embodiments, the system comprises more than one source of an additive, each source configured to introduce a separate additive into the biofluid. Thus, the system may comprise a source of a second additive configured to introduce the second additive into the biofluid. In certain embodiments, the second additive may be an additive capable of altering or regulating at least one of size of the target cells, size of the non-target cells, compressibility of the biofluid, compressibility of the target cells, compressibility of the non-target cells, aggregation potential of the target cells, aggregation potential of the non-target cells, density of the biofluid, density of the target cells, density of the non-target cells, or any combination thereof.

In some embodiments, the source of the additive may be substantially free of a reagent capable of altering or regulating one or more of size of the target cells, size of the non-target cells, compressibility of the biofluid, compressibility of the target cells, compressibility of the non-target cells, aggregation potential of the target cells, and aggregation potential of the non-target cells, as previously discussed. The additive may further be capable of altering or regulating at least one of density of the biofluid, density of the target cells, density of the non-target cells, or any combination thereof.

The system may further comprise at least one acoustic transducer coupled to a wall of the microfluidic separation channel. The acoustic transducer may be positioned to apply a standing acoustic wave transverse to the microfluidic separation channel. In some embodiments, the acoustic transducer is capable of emitting acoustic energy that drives cells and/or particles to a pressure node or anti-node. The acoustic transducer may comprise a piezoelectric vibrating element configured to emit acoustic energy. The denser and larger particles and cells may migrate towards the center of the separation channel in response to the acoustic energy emitted by the piezoelectric transducer. The acoustic transducer may be configured to provide standing acoustic waves having a wavelength that is twice as long as the microfluidic separation channel width.

In some embodiments, the methods disclosed herein and the acoustic transducer may be configured to emit acoustic energy between about 1.0 MHz and about 4.0 MHz. For instance, the acoustic transducer may emit acoustic energy between about 1.5 MHz and about 3.5 MHz or between about 1.0 MHz and about 2.0 MHz. In some embodiments, the methods disclosed herein may comprise supplying the acoustic transducer with a voltage of between about 5 V and about 100 V, for example, a voltage of between about 5 V and about 50 V, between about 8 V and about 36 V, between about 12 V and about 36 V, or between about 16 V and about 24 V, for example, about 5 V, 8 V, 12 V, 16 V, 24 V, 36 V, 50 V, or 100 V. The voltage may be selected to increase recovery of target cells or non-target cells in the output suspension. In some embodiments, the voltage may be selected responsive to a measured parameter.

The microfluidic separation channel may further comprise one or more heat sinks configured to dissipate heat generated by the acoustic transducer. The heat sink may be configured to dissipate enough heat from the acoustic transducer to prevent the transducer from warming fluids flowing through the separation channel. In some embodiments, the heat sinks comprise thermoelectric coolers. In some embodiments, the system includes fluidic lines that flow into the heat sink to provide fluidic cooling to the heat sink.

The system may comprise a control module configured to introduce the biofluid into the microfluidic separation channel. For example, the control module may be configured to direct a pump to flow the pretreated biofluid into the at least one inlet of the microfluidic separation channel. The control module may be configured to control or regulate the flow rate of the biofluid. In some embodiments, the control module may control or regulate the flow rate of the biofluid responsive to a measured parameter of the source of the biofluid, within the microfluidic separation channel, or of the output suspension. The control module may direct the biofluid responsive to automatic or manual control in accordance with the methods disclosed herein.

The system may comprise a control module configured to introduce a predetermined volume of the additive into the biofluid to produce the pretreated biofluid. The control module may comprise or be in electrical communication with a pump or flow meter. For example, the control module may be configured to direct a pump to introduce the additive into the biofluid. The control module may introduce the predetermined volume of the additive responsive to automatic or manual control in accordance with the methods disclosed herein. For example, the control module may introduce the predetermined volume of the additive periodically, as needed, and/or responsive to a timer. The control module may introduce the predetermined volume of the additive responsive to a manual indication.

Systems that comprise more than one microfluidic separation channel may comprise one acoustic transducer coupled to each microfluidic separation channel or one or more acoustic transducers coupled to a collection of microfluidic separation channels.

In some embodiments, the system comprises at least two microfluidic separation channels. The at least two microfluidic separation channels may be arranged in a parallel arrangement downstream of the source of the biofluid. The system may be scaled to include sufficient microfluidic separation channels to achieve a throughput of 1 ml/min, for example, exceeding 1 ml/min.

The system may further comprise a manifold configured to distribute biofluid to the at least two microfluidic separation channels arranged in parallel. The manifold may be configured to receive a biofluid or pretreated biofluid sample and distribute the sample to downstream microfluidic separation channels. The manifold may distribute the biofluid sample substantially evenly, in a ratio selected in accordance with a predetermined protocol, or in a ratio selected responsive to a measured parameter. In some embodiments, the manifold may be configured to continuously receive and distribute fluid, and in other embodiments the manifold may be configured to receive and distribute fluid in batches. The manifold configured to receive and distribute fluid in batches may be on a regular timer or may distribute fluid batches as it receives sufficient fluid.

In some embodiments, the manifold is configured to distribute the biofluid in response to the input biofluid load on the system. In some embodiments, the input biofluid load comprises between about 1 mL to about 1 L of fluid. In some embodiments, the input biofluid load on the system may have a flow rate of between about 0.1 mL/min to about 10 mL/min. Each microfluidic separation channel may be configured to receive flow rates of between about 0.1 mL/min to about 0.5 mL/min. The system may further comprise at least one sensor configured to measure an input biofluid load on the system. The input biofluid load sensor may be in electrical communication with the manifold, such that the manifold may distribute the biofluid to the two or more microfluidic separation channels in response to the measurement of the input biofluid load received from the input biofluid load sensor.

In some embodiments, the system further comprises at least one sensor configured to measure at least one parameter of the input biofluid. The sensor may be configured to measure at least one parameter of the pretreated biofluid. For instance, the biofluid sensor may be configured to measure an input biofluid load on the system. The sensor may be configured to measure at least one of flow rate, density of the biofluid, HCT % of the biofluid, concentration of target cells, concentration of non-target cells, concentration of cluster-forming cells, or concentration of non-target cell clusters in the biofluid or pretreated biofluid. In some embodiments, the biofluid sensor is configured to measure optical transmission or absorption of the biofluid at a predetermined optical wavelength. The at least one biofluid sensor may be positioned at the system input and configured to measure parameters from the input biofluid load or may be positioned within the source of the biofluid and configured to measure parameters from the biofluid or pretreated biofluid.

The system may further comprise a control module in electrical communication with the input or biofluid sensor. The control module may further be in electrical communication with the source of additive and configured to introduce a predetermined volume of the additive into the biofluid in response to the measurement of the at least one parameter of the input biofluid or pretreated biofluid. The control module may be configured to control flow rate of the biofluid and/or pretreated biofluid in response to the measurement of the at least one parameter of the input biofluid or pretreated biofluid.

The system may comprise a source of the cluster-forming cells in fluid communication with the source of the biofluid. In some embodiments, the control module may be in electrical communication with the source of the cluster-forming cells. The control module may be configured to introduce a predetermined amount of the cluster-forming cells into the biofluid in response to at least one parameter of the input biofluid or pretreated biofluid, for example, a measurement of the at least one parameter. In some embodiments, the control module may be configured to introduce a predetermined amount of the cluster-forming cells into the biofluid in response to a concentration of the cluster-forming cells already present in the biofluid and/or a concentration of the target cells or the non-target cells in the biofluid.

According to certain embodiments, the system further comprises at least one sensor configured to measure a parameter of an output suspension. The output suspension may be target cell enriched fluid or target cell depleted fluid exiting the microfluidic separation channel through the at least one outlet, or product or waste exiting the system. The sensors may measure at least one of flow rate, HCT %, concentration of target cells, concentration of non-target cells, or concentration of non-target cell clusters in the output suspension. In some embodiments, the sensors may measure at least one of density of the output suspension, density of the target cells, density of the non-target cells, size of the target cells, size of the non-target cells, compressibility of the output suspension, compressibility of the target cells, compressibility of the non-target cells, and concentration of the additive in the output suspension. In some embodiments, the sensors may measure optical transmission or absorption of the output suspension at a predetermined wavelength.

According to certain embodiments, the system further comprises at least one sensor configured to measure a parameter within the microfluidic separation channel. The device sensor may be configured to measure at least one of temperature, pressure, or acoustic energy within the microfluidic separation channel. Acoustic energy may be measured in the form of power, voltage, or frequency delivered to the acoustic transducer.

The control module may be in electrical communication with the acoustic transducer and configured to alter or regulate at least one input parameter of the acoustic transducer. The control module may be configured to direct the acoustic transducer to apply the acoustic energy to the microfluidic separation channel. For instance, the control module may alter or regulate the power, voltage, or frequency delivered to the acoustic transducer. The control module may direct the acoustic transducer responsive to automatic or manual control in accordance with the methods disclosed herein.

The control module may be in electrical communication with the device sensor or the output suspension sensor. In some embodiments, the control module may be configured to direct the acoustic transducer in response to a measurement of a parameter of the output suspension or within the microfluidic separation channel. The control module may further shut on or off the acoustic transducer in response to a measurement of a parameter of the output suspension. For instance, the control module may act in response to a measurement of flow rate, HCT %, concentration of target cells, concentration of non-target cells, or concentration of non-target cell clusters in the output suspension. The control module may act in response to a measurement of temperature, pressure, or acoustic energy within the microfluidic separation channel.

The control module may further be in electrical communication with the source of additive and configured to introduce a predetermined volume of the additive into the biofluid in response to the measurement of the at least one parameter of the output suspension or within the microfluidic separation channel. The control module may be configured to control flow rate of the biofluid and/or pretreated biofluid in response to the measurement of the at least one parameter of the output suspension or within the microfluidic separation channel.

The control module in communication with any sensor may be the same or different from the control module in communication with any other sensor. In some embodiments, any control module may be designed to act in response to a measurement from any sensor within the system. For instance, the control module configured to introduce a predetermined volume of additive into the biofluid may further be in electrical communication with the output suspension sensor or input biofluid load sensor and configured to act in response to a measurement received therefrom. In another embodiment, the control module configured to be in electrical communication with the acoustic transducer may also be in electrical communication with other sensors and configured to act in response to a measurement received from the biofluid load sensor or the biofluid sensor.

The system may further comprise a source of a second fluid in fluid communication with the at least one inlet of the at least one microfluidic separation channel. The source of the second fluid may be a vessel, tank, or chamber in fluid communication with the microfluidic separation channel, the source of the biofluid, or a line connecting the source of the biofluid with the at least one inlet of the microfluidic separation channel. The source of the second fluid may be configured to introduce the second fluid into the microfluidic separation channel or into the biofluid. In some embodiments, the biofluid and the second fluid flow in substantially parallel, substantially laminar flow, as previously discussed. The second fluid may be any fluid, as previously discussed.

In some embodiments, the system may further comprise a first and second collection channel in fluid communication with the at least one outlet of the microfluidic separation channel. The collection channel may be a fluid line configured to deliver output suspension to a vessel, recycle line, or fluidly connectable with an intraluminal line configured to deliver output suspension to a subject. A collection vessel may be in fluid communication with the first or second collection channel. The collection vessel may be used to store, freeze, heat, or otherwise keep output suspension.

According to certain embodiments, the system further comprises a recycle line. In some embodiments, the recycle line is a line or channel configured to deliver output suspension back to the source of the biofluid for a second pass separation. The recycle line may be configured to deliver output suspension back to the at least one inlet of the microfluidic separation channel. The output suspension that is recycled may be target cell enriched fluid or target cell depleted fluid.

In some embodiments, the system comprises a post-treatment chamber. The post-treatment chamber may be configured to post-treat output suspension to produce a post-treated fluid, physiologically acceptable fluid, or therapeutic fluid, as previously described.

The system may comprise one or more pumps to direct the biofluid through the system. The one or more pumps may be an infusion pump configured to generate sufficient pressure to force the biofluid through the system. In some embodiments, the pump generates sufficient pressure to introduce the output suspension into the recipient subject through the intraluminal line.

The system may be connectable to more than one intraluminal line to produce an in-line system for separation of cells. For instance, the system may be connectable to an intraluminal line configured to extract biofluid from a donor subject and deliver it to the source of the biofluid for processing. The system may be connectable to an intraluminal line configured to deliver an output suspension, for example target cell enriched fluid or target cell depleted fluid, to the recipient subject. In some embodiments, the recipient subject may be the same as the donor subject, and the biofluid processing is performed in line and in real time. In some embodiments, the recipient subject and the donor subject may be different from one another.

In some embodiments, the system comprises more than one microfluidic separation channel arranged in series. The more than one microfluidic channel in series may be configured to separate target cells from non-target cells in consecutive separation channels to produce a fluid with high target cell purity. In some embodiments, the more than one microfluidic separation channel in series is configured to deliver target cell enriched fluid to downstream microfluidic separation channels. In alternate embodiments, the more than one microfluidic separation channel in series is configured to deliver target cell depleted fluid to downstream microfluidic separation channels. In some embodiments, the microfluidic separation channels in series are stacked to process relatively larger volumes of biofluid. The stacked configuration allows branched outlets of the separation channel to be easily connectable to branched inlets of a downstream separation channel.

In accordance with another aspect, there is provided a kit for separation of target cells from non-target cells. The kit may comprise at least one microfluidic separation channel connected to an acoustic transducer, a source of an additive fluidly connectable to the at least one inlet of the microfluidic separation channel, and instructions for use. The at least one microfluidic separation channel may be configured to separate target cells from non-target cells, as previously described herein. The source of the additive may be a vessel, chamber, or channel, as previously discussed herein and may comprise at least one additive, as previously discussed herein. The kit may further comprise any component of the system described herein, connectable to the microfluidic separation channel. For instance, according to certain embodiments, the kit may further comprise a collection channel, a collection vessel, a manifold system, a sensor, a control module, an intraluminal line, a pump, a post-treatment chamber, or fluid lines to fluidly connect the components of the kit.

The kit may comprise a collection channel fluidly connectable to one of the first outlet and the second outlet of the microfluidic separation channel. The kit may comprise a collection vessel fluidly connectable to the collection channel. The kit may comprise a collection channel fluidly connectable to the first outlet and configured to recycle target cell enriched fluid or target cell depleted fluid to the microfluidic separation channel. The kit may comprise an intraluminal line fluidly connectable to one of the microfluidic separation channel and the first or the second outlet. The kit may comprise more than one microfluidic separation channel fluidly connectable to the source of the biofluid in parallel or in series. The kit may comprise one or more sensors or control modules connectable to the microfluidic separation channel.

The kit may include instructions to collect a biofluid, pretreat the biofluid by introducing a predetermined volume of additive into the source of the biofluid, flow the pretreated biofluid through the microfluidic separation channel, and apply acoustic energy to the separation channel. The kit may include instructions to provide a biofluid, pretreat the biofluid by introducing a predetermined volume of the additive into the biofluid to form a pretreated biofluid comprising the target cells and the non-target cell clusters, flow the pretreated biofluid into the at least one inlet of the microfluidic separation channel, and apply acoustic energy to the microfluidic separation channel to separate the target cells from the non-target cell clusters.

The kit may further comprise instructions to control the power, voltage, or frequency of the acoustic transducer to alter or regulate the HCT %, concentration of target cells or concentration of non-target cells in the output suspension, as previously discussed herein. For instance, the kit may comprise instructions to regulate the output suspension HCT % to be less than about 10%. The kit may comprise instructions to perform any step or collection of steps from the method of separating target cells from non-target cells.

In accordance with another aspect, there is provided a method of facilitating separation of target cells from non-target cells. The method may comprise providing at least one of a microfluidic separation channel, an acoustic transducer, a source of an additive fluidly connectable to the at least one inlet of the microfluidic separation channel, and instructions for use. The method may comprise providing a biofluid. In certain embodiments, the method may comprise providing a source of cluster-forming cells. The method may comprise providing a control module and/or at least one sensor.

The method may comprise providing pretreat the biofluid by introducing a predetermined volume of the additive into the biofluid to form a pretreated biofluid comprising the target cells and the non-target cell clusters, flow the pretreated biofluid into the at least one inlet of the microfluidic separation channel, and apply acoustic energy to the microfluidic separation channel to separate the target cells from the non-target cell clusters. The method may comprise providing instructions to electrically connect the control module to at least one sensor, the acoustic transducer, the source of the additive, and/or the source of the biofluid. The method may comprise programming the control module to operate as described herein.

The function and advantages of the embodiments discussed above and other embodiments of the invention can be further understood from the description of the figures below, which further illustrate the benefits and/or advantages of the one or more systems and techniques of the invention but do not exemplify the full scope of the invention.

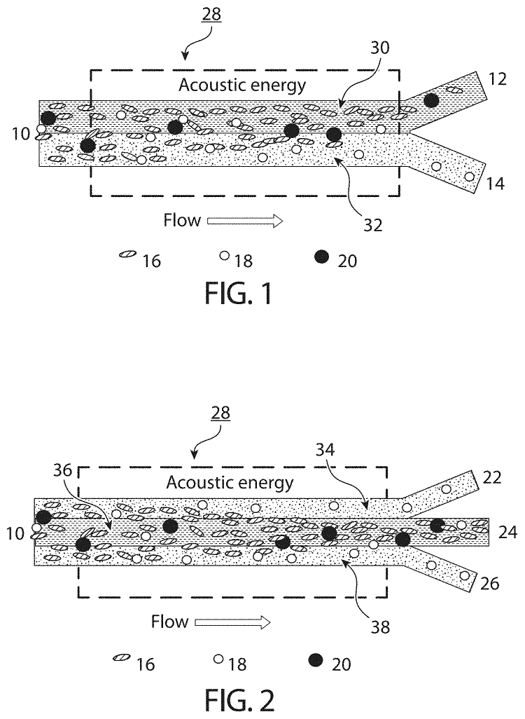

FIG. 1 is an exemplary concept schematic drawing illustrating the general principles of microfluidic acoustic separation. As shown in FIG. 1, a biofluid comprising target cells 18 and non-target cells 16 and 20 is flowed through microfluidic separation channel 28, through the inlet 10. Acoustic energy is applied to the separation channel 28 within the illustrated dotted line rectangle. Acoustic energy may be applied by attaching a piezoelectric transducer (not shown) to one wall of the separation channel. Target cells 18 accumulate within primary stream 32 and exit the separation channel 28 through first outlet 14. Target cell enriched fluid exits the first outlet 14. Non-target cells 16 and 20 accumulate within secondary stream 30 and exit the separation channel through second outlet 12. The non-target cells 18 and 20 are contained in a waste fluid. In some embodiments, the target cell enriched fluid within the primary stream 32 is collected.

FIG. 2 is an exemplary concept schematic drawing illustrating an alternate microfluidic acoustic separation. As shown in FIG. 2, the biofluid comprising target cells 18 and non-target cells 16 and 20 is flowed through the microfluidic separation channel 28 through inlet 10. In the embodiment exemplified in FIG. 2, target cells 18 essentially accumulate within two primary streams, 34 and 38, at the periphery of the separation channel 28, upon being subjected to the acoustic energy. Non-target cells 16 and 20 essentially accumulate within the central secondary stream 36. The primary streams 34 and 38 (target cell enriched fluid) exit the separation channel 28 through peripheral first outlets 22 and 26, while the secondary stream 36 (waste fluid) exits the separation channel 28 through second outlet 24. In this exemplary embodiment, non-target cells 16 and 20 are more susceptible to the acoustic energy, so they travel rapidly to the central region (secondary stream 36) of the separation channel 28, while the target cells 18 experience a weaker force from the acoustic energy and remain in the peripheral region of the separation channel 28 (primary streams 34 and 38).

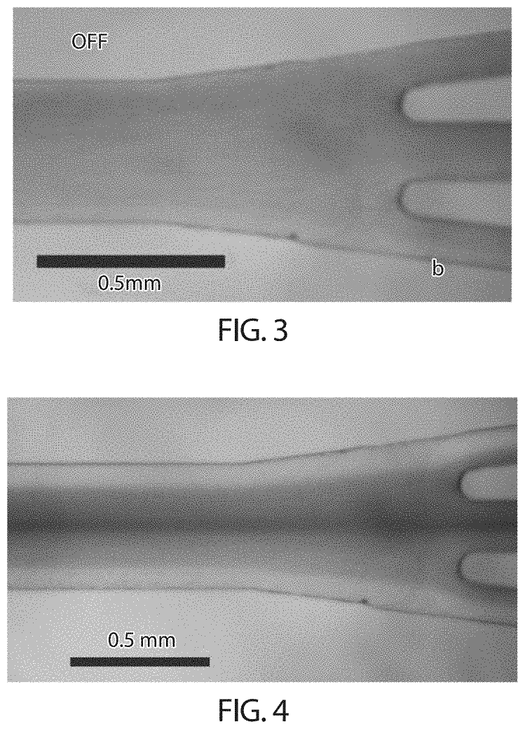

FIG. 3 and FIG. 4 are microscopic images of the downstream end of a microfluidic separation channel. In FIG. 3, the microfluidic separation channel is receiving no acoustic energy. As shown in the image, a homogeneous cell suspension is flowing through the channel with no separation. In FIG. 4, the microfluidic separation channel is receiving acoustic energy. Non-target cells, shown as the darker shade, can be seen traveling through the center stream, while target cells (not individually visible in the images) travel through the outer streams. The separation as seen in FIG. 4 is much greater than that seen in FIG. 3.

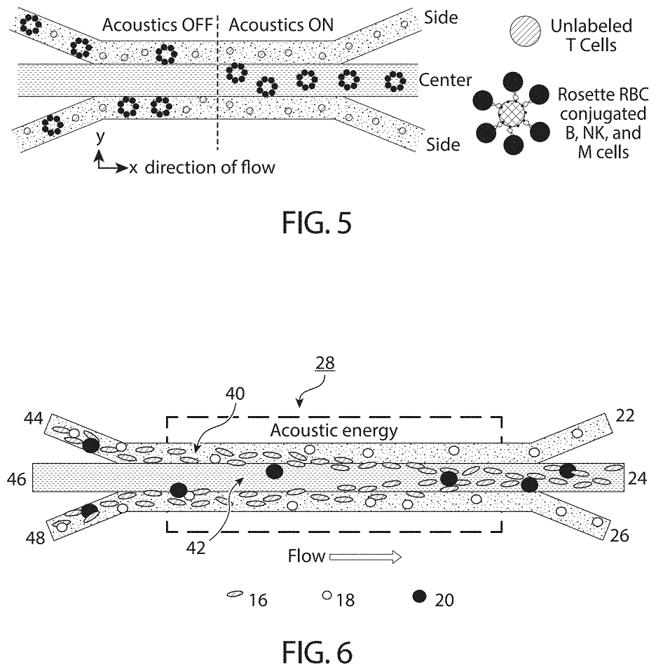

In accordance with certain embodiments disclosed herein, target cells or non-target cells may be formed into cell clusters, as shown in FIG. 5. The clusters may be formed by introducing an additive comprising a cocktail of antibodies selected to bind certain cells. Unbound cells and cell clusters may be driven to pressure nodes or anti-nodes by acoustic energy generally as shown in the schematic drawings of FIGS. 1-2. Thus, as shown in the exemplary embodiment of FIG. 5, a target leukocyte (here T cells) may be separated from non-target cells (here B cells, NK cells, and monocytes) using antibodies selected to bind the non-target cells. Acoustic energy may be applied to deplete the non-target cell clusters from the blood sample.

In certain embodiments, for example, as shown in exemplary concept schematic drawing of FIG. 6, a second fluid 42 may be flowed through the microfluidic separation channel 28 with pretreated biofluid 40, in essentially parallel flow. The second fluid 42 enters the microfluidic separation channel 28 through central inlet 46, while pretreated biofluid 40 enters the microfluidic separation channel 28 through peripheral inlets 44 and 48. The second fluid 42 does not comprise cells as it enters the separation channel 28. Non-target cells 16 and 20 are driven towards the center stream by the applied acoustic energy and exit the separation channel through waste outlet 24. Target cells 18 are essentially buoyant within the microfluidic separation channel 28, and are not driven to the central stream. The estimated recovery in the exemplary embodiment of FIG. 6 is calculated to be about 70%. Comparatively, the estimated recovery in an embodiment without introducing a second fluid, such as the one exemplified in FIG. 2, is about 65%.

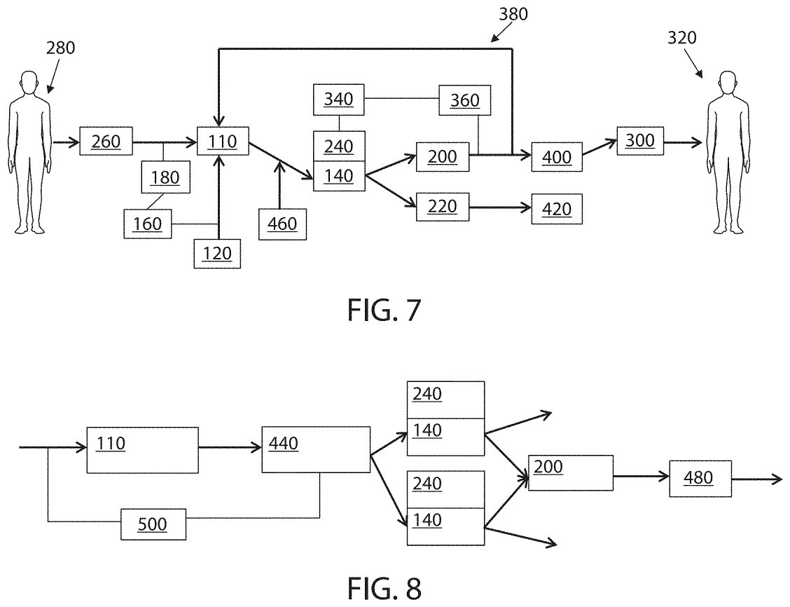

As shown in FIG. 7, according to certain embodiments, a system for microfluidic separation of target cells and non-target cells in a biofluid may comprise a source of a biofluid 110, a source of an additive 120, and a microfluidic separation channel 140 coupled to an acoustic transducer 240. The system may further comprise a sensor 180 configured to measure a parameter of an input biofluid and a sensor 360 configured to measure a parameter of a primary stream. The sensors may be electrically connected to control modules 340 and 160, such that control module 340 is configured to alter or regulate an input parameter of the acoustic transducer 240 and the control module 160 is configured to introduce a predetermined volume of the additive into the biofluid.

The system may further comprise intraluminal line 260 fluidly connected to donor subject 280 and second intraluminal line 300 fluidly connected to recipient subject 320. Recipient subject 320 and donor subject 280 may be the same subject. The microfluidic separation channel 140 may separate pretreated biofluid into a primary stream and a secondary stream, such that the primary stream comprising target cells (target cell enriched fluid) is directed to primary stream collection channel 220 and the secondary stream comprising non-target cells (target cell depleted fluid) is directed to secondary stream collection channel 220. The primary stream may be recycled back to the source of the biofluid 110 through recycle line 380 or may be post-treated in post-treatment chamber 400. In some embodiments, the post-treatment chamber 400 is fluidly connected to the intraluminal line 300. The secondary stream may be collected in collection vessel 420. The system may further comprise a source of a second fluid 460 fluidly connected to the microfluidic separation channel 140.

Turning to FIG. 8, the system for microfluidic separation of target cells and non-target cells in a biofluid may further comprise two or more microfluidic separation channels 140. In the embodiment as shown, each microfluidic separation channel 140 is coupled to an acoustic transducer 240, however the system may comprise one acoustic transducer 240 coupled to more than one microfluidic separation channel 140. The two or more microfluidic separation channels 140 may be fluidly connected to a manifold 440, which may be fluidly or electrically connected to a sensor 500. The manifold 440 may be configured distribute the biofluid to the microfluidic separation channels 140 in response to a measurement received from the sensor 500 of an input biofluid load upstream of the biofluid source 110. In some embodiments, the system comprises a collection channel 200 downstream from the microfluidic separation channels 140 configured to collect the primary stream from the microfluidic separation channels 140. The system may further comprise a collection vessel 480 downstream from the collection channel 200.

### EXAMPLES

### Example 1: Comparison Between Target Cells—Lymphocyte and Monocyte Separation Ratio

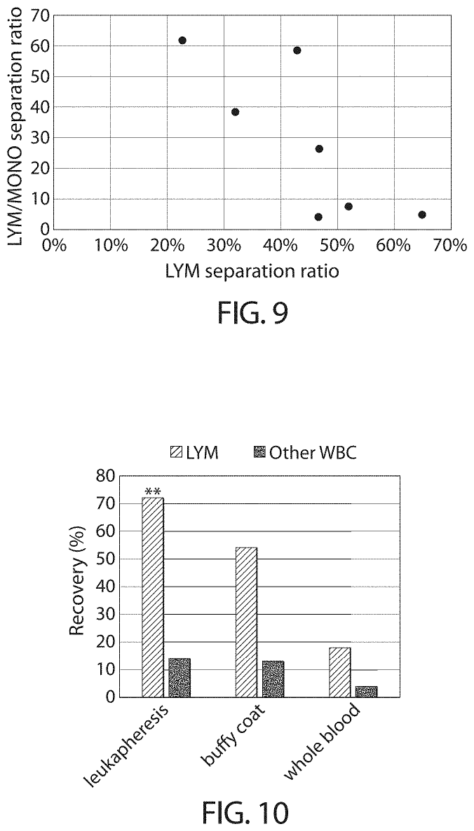

Biofluid comprising lymphocytes and monocytes was flowed through a microfluidic separation channel and subjected to acoustic energy. The lymphocyte separation ratio was calculated as previously discussed. The monocyte separation ratio was compared to the lymphocyte separation ratio. The results are shown in the graph of FIG. 9. The data suggest that there is a differential separation between monocytes and lymphocytes. The results are significant because other separation processes, for example centrifugation, do not separate lymphocytes from monocytes. Accordingly, systems and methods disclosed herein allow for differential separation between cell types, including between different classes of leukocytes.

### Example 2: Comparison Between Biofluids—Lymphocyte Purity and Recovery from Leukapheresis Product, Buffy Coat, and Whole Blood

Acoustic separation of lymphocytes from biofluid samples was performed, generally as described above. The biofluid samples included leukapheresis product, blood buffy coat, and whole blood. Generally, leukapheresis product comprises the highest ratio of leukocytes to other cells, blood buffy coat comprises a mid-range ratio of leukocytes to other cells, and whole blood comprises the lowest ratio of leukocytes to other cells. Accordingly, as expected, lymphocyte recovery (percentage of lymphocyte in product to lymphocyte in biofluid sample), and lymphocyte purity (as a fraction of lymphocyte to total leukocyte concentration) is greatest when the biofluid is selected to be leukophoresis product, of the three example biofluids.

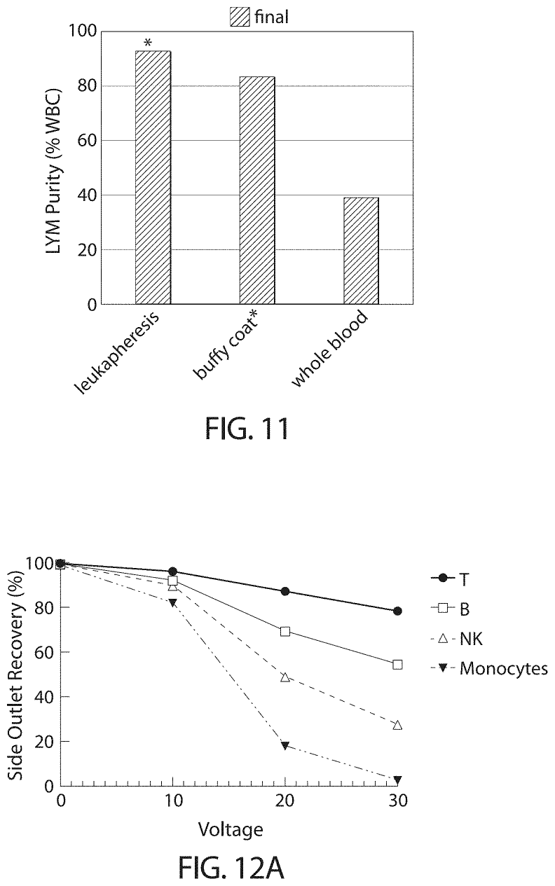

As shown in the results presented in the graphs of FIGS. 10 and 11, lymphocyte recovery from leukophoresis product, buffy coat, and whole blood is 71%, 54%, and 18%, respectively. Lymphocyte purity in these samples was high, at 93%, 83%, and 39%, respectively. Furthermore, the separation provided erythrocyte reduction (percentage of erythrocyte reduced from the biofluid sample) of about 94%, depending on the recovery goal. Accordingly, systems and methods for cell separation, as disclosed herein, may effectively recover and purify biofluid samples of various purities with a first pass acoustic separation process.

### Example 3: Enrichment of Selected Leukocytes from Blood Using a Cocktail of Antibodies Binding Non-Target Cells to Cluster-Forming Cells

Cancer cell therapies often require isolation of large quantities of target leukocytes, such as T cells, B cells, or NK cells, from blood samples. Acoustophoresis is effective at depleting monocytes, neutrophils, and erythrocytes from blood samples containing leukocytes without additional reagents. However, without reagents it is challenging to separate classes of lymphocytes from one another.

Here, improved enrichment of a target class of leukocytes from a blood sample was achieved by introducing a cocktail of antibodies selected to bind non-target leukocytes to erythrocytes, forming non-target cell clusters. The non-target cell-clusters were shown to be capable of being separated from the target leukocytes by acoustophoresis.

FIG. 5 is a schematic drawing showing acoustic separation of a target leukocyte, here T cells, using antibodies selected to bind non-target cells, here B cells, NK cells, and monocytes, to deplete the non-target cells from the blood sample.

Fresh leukapheresis product from healthy human donors was obtained (Leukopak, distributed by Hemacare® Cellular Products, Los Angeles, Calif.). White blood cell (WBC) and red blood cell (RBC) counts were measured with a hematology analyzer (Sysmex, Kobe, Hyogo, Japan). A polystyrene microchannel affixed to a piezoelectric transducer was used for microfluidic acoustic separation.

The leukapheresis product was resuspended in media at 50×10⁶ WBC/mL retaining all WBCs and RBCs. The resuspended leukapheresis was incubated with the antibody cocktail (RosetteSep™, distributed by StemCell Technologies, Vancouver, Calif.) for about 60 min and processed on the microchannel separation device. Control resuspended leukapheresis product was processed on the microchannel separation device without incubation with the antibody cocktail. To test impact of RBC to WBC ratio, some samples were spiked with supplementary RBCs pelleted from whole blood from the same donor.

Samples were directed through the microchannel device at a sample flow rate of 100 μL/min through the side inlets and a media was directed through the microchannel device at flowrate of 400 μL/min through the center inlet. To determine the appropriate transducer voltage (to achieve both high purity and high recovery) and account for donor variation in leukopak and device efficiency, several voltages as applied to the piezoelectric transducer were tested for each treatment condition. While operating the device, the displacement of the RBCs or rosettes was observed under bright field microscopy. At very low voltages, purification performance was poor as most waste cells were not displaced and remained in the side streams. At excessively high voltages, recovery of target cells fell off as both waste and target cells were depleted from the sides. By taking a broad range of voltages, the optimal voltage was found for each test sample.

Flow cytometry was performed to determine the output purity of CD3+ T cells, CD19+ B cells, CD14+ monocytes, and CD56+NK cells as a percent of CD45+ leukocytes. Purity of target cells in the side fraction (product) was measured at each applied voltage. Absolute cell numbers from output fractions were calculated using an imaging cytometer (Celigo, San Mateo, Calif.). Recovery was defined as the percent of each cell type retained in the side outlets out of the total collected from side and center outlets. Rosette ratio (RR) indicates predicted number of RBCs bound to a single PBMC.

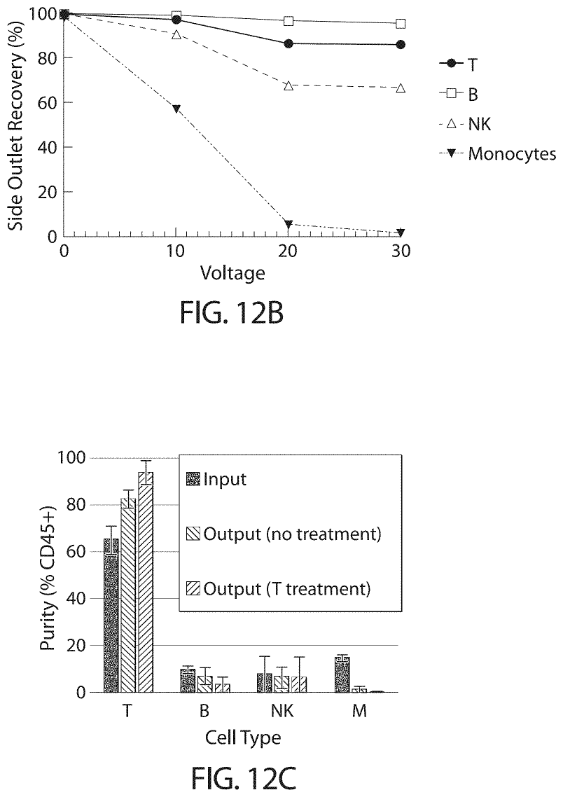

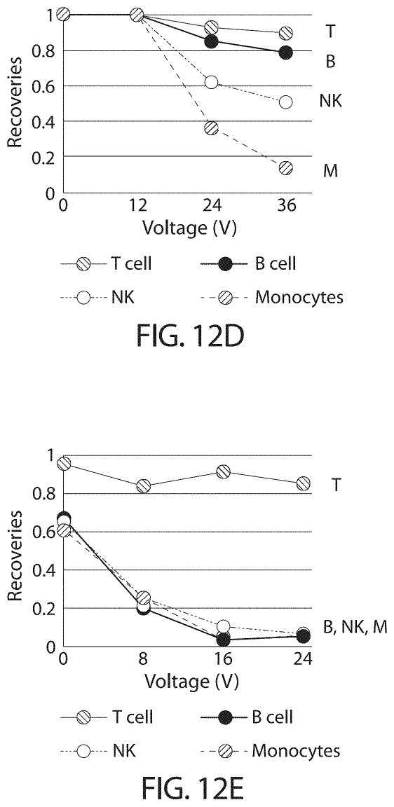

Addition of the antibody cocktail improved acoustic T-cell purification, achieving an average output purity of 94% CD3+ in the product from three donors, and reaching 99% CD3+ in the product from one donor. The graphs of FIGS. 12A-12B show recovery of cell types for one representative separation across increasing voltage. In both the antibody cocktail treated (FIG. 12A) and untreated (FIG. 12B) samples, increasing voltage reduced recovery of all cell types. Compared with the untreated sample, the antibody cocktail treatment specifically reduced the recovery (i.e., increases desired depletion) of the non-target cells, but caused only minor reduction of the recovery of the target T cells. As shown in FIG. 12C, addition of the antibody cocktail improved acoustic T-cell purification from an input sample of about 64% T-cell population to about 94% T-cell purity, as compared to an untreated sample which achieved about 82% T-cell purity.

Recovery of non-target cells in the output decreased with increasing voltage applied to the piezoelectric transducer in samples treated with the antibody cocktail (FIG. 12D) and untreated samples (FIG. 12E). However, recovery was markedly reduced for all non-target cells (B cells, NK cells, and mononuclear cells) in the treated sample. At the optimum voltage, T cell recovery was 92%.

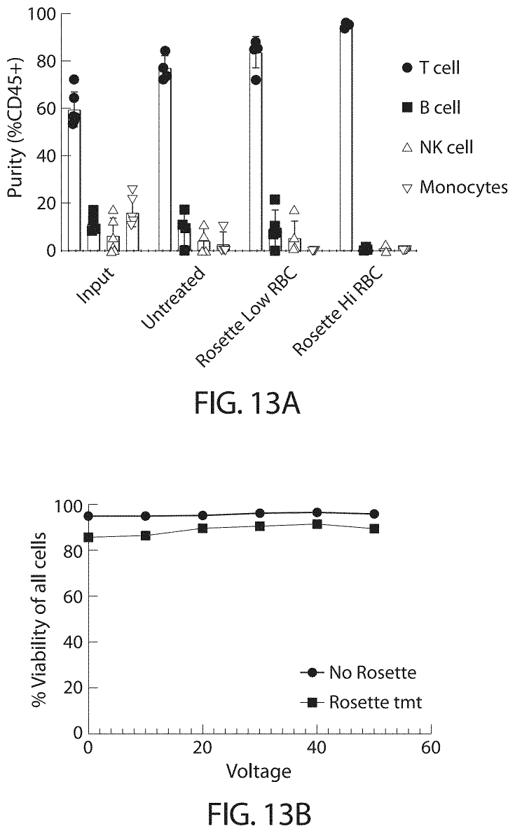

FIG. 13A is a graph showing purity of cell types at the voltage that achieved the desired characteristics of both high purity and high recovery. Acoustic separation of untreated cells effectively depleted monocytes causing a slight enrichment in T cell purity. However, B cells and NK cells were only marginally depleted. Addition of the antibody cocktail reagent to leukapheresis product (low RBC) further enriched T cells. Under the preferred conditions recovery of T cells was greater than 90%.

Even with high enrichment, some product samples did not meet purity likely required for clinical application. To further increase purity, high RBC content was tested. Certain samples contained naturally high RBC count. Other samples were spiked with additional RBCs, as previously described. The higher RBC count resulted in purity approaching 95%. T cell recovery data accompanying the purity results in FIG. 13A are shown in Table 1. Viability of all cells was generally greater than 90% (FIG. 13B), confirming acoustic separation is not detrimental to cell viability.

TABLE 1
Average T cell recovery across 6 donors. Antibody cocktail
at low RBC was tested in only 5 of 6 donors and antibody
cocktail in high RBC was tested in 3 of 6 donors.

| | | Mean | |
|---|---|---|---|
| | Num. | Recovery | St. Dev. |
| Treatment | Donors | (%) | (%) |
| Untreated | 5/6 | 70.4 | 26.1 |
| Antibody w/low RBC | 5/6 | 67.4 | 16.5 |
| Antibody w/high RBC | 3/6 | 90.5 | 3.73 |

To confirm that the antibody cocktail separation is effective on other cell types, a similar separation was performed to select for B cells by introducing an antibody cocktail selected to conjugate all mononuclear cells (T cells, NK cells, and monocytes) except B cells. The results are presented in the graphs of FIGS. 14A-14B. As shown in the graph of FIG. 14A, the separation resulted in about 16× enrichment of B cells. As shown in the graph of FIG. 14B, antibody cocktail treatment at low RBC count increased the purity of B cells significantly. The purity was further increased by antibody cocktail treatment at high RBC count, resulting in an increase of B cell purity from an input of 5% to an output of 95%, with B cell recovery of 85%.

Accordingly, antibody cocktail treatment selected to form cell clusters of non-target cell types with RBCs was shown to be effective at improving acoustic separation. Cells enter the device in the side inlet and experience acoustic forces that direct the cells toward the center of the channel. The factors that affect the drive by the acoustic force are volume, density and compressibility. RBCs, especially because of their higher density, tend to undergo greater acoustic forces than lymphocytes. By binding waste lymphocytes (non-target cells) to RBCs and forming clusters, the acoustic forces experienced by the clusters become higher than an unbound cell. The energy required to focus clusters to the center is lower than the energy required to focus target lymphocytes. At an intermediary acoustic energy, clusters containing waste (non-target) cells can be depleted from the side streams, while the unbound target cell type remains. The data presented herein demonstrate that the methods are effective at purifying both T cells, which are natively high in purity, and B cells, which are natively low in purity.

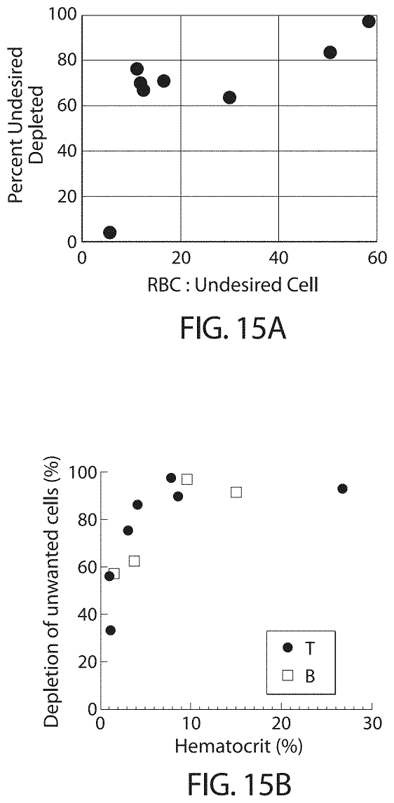

Because it was observed that the resulting output purity of target T cells was sometimes limited by the number of RBCs in the leukapheresis product (typically 1-3% hematocrit) to about 85% purity, RBCs from donor matched whole blood was spiked into the leukapheresis product. After acoustic separation, 95% or more purity for T cells was achieved (as previously reported in FIG. 13A). The ratio of RBCs to unwanted WBCs was measured in 8 samples, spiked with additional RBCs or not, to establish requirements for effective purification. As shown in the graph of FIG. 15A, a higher ratio of RBCs improved purification. Thus, percent of depleted cells was shown to increase with increasing ratio of RBCs to unwanted WBCs. It is hypothesized that still higher ratios, however, would interfere with the purification and therefore that there is an optimum ratio or range of ratios for acoustophoretic separation.

To further analyze this result, the results were normalized for donor variability in input cell populations by calculating the total depletion of non-target cells as a fraction of the total non-target cells in the input sample. FIG. 15B is a graph showing the results of the normalization analysis as a function of hematocrit for both T cell and B cell depletion. As shown in FIG. 15B, a positive correlation was found with hematocrit for both target cells. Thus, the output purity was observed to be a function of hematocrit of the leukapheresis product for the tested samples. Since hematocrit levels can be controlled (for example, during leukapheresis collection by tuning apheresis parameters, it is suggested that the separation methods disclosed herein may be seamlessly integrated into the current practices for sourcing patient T cells for autologous therapy.

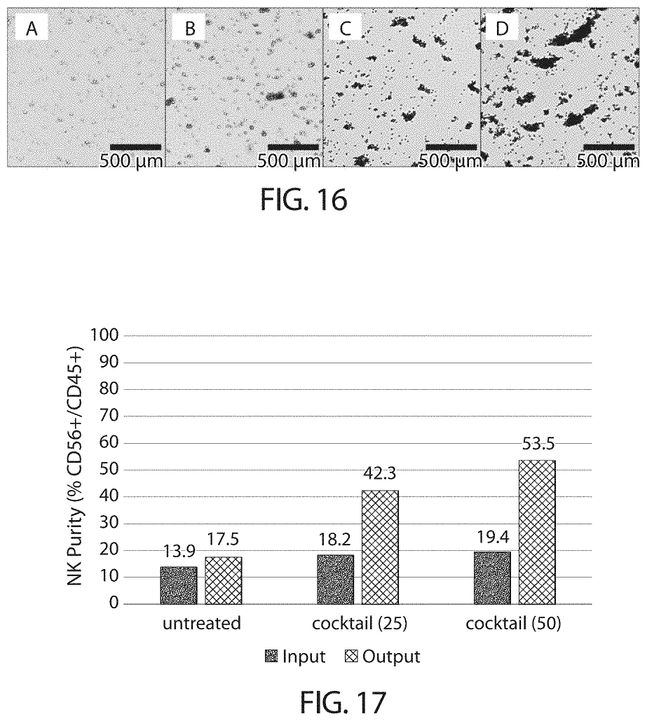

Additionally, the size of the cell clusters in leukapheresis product was observed by visual inspection under brightfield microscopy (FIG. 16). FIG. 16 includes micrograph images of leukapheresis product after addition of the antibody cocktail for T cell isolation. Images A-D contain approximately 10, 20, 30 and 60 RBCs per WBCs, respectively. The clusters were shown to increase with an increasing ratio of RBCs to WBCs. The size of the clusters appeared widely variable, as observed qualitatively. Thus, larger clusters may be formed with increasing ratio of RBCs to WBCs. However, it is hypothesized that there is a critical ratio that would interfere with separation by forming clusters that are too large for the microfluidic channel and therefore there is an optimum ratio or range of ratios for microfluidic separation.

Accordingly, cell cluster formation linking non-target cells to erythrocytes enables purification of selected target cells, T cells or B cells, by acoustophoresis without addition of synthetic particles or a property-altering additive. In certain embodiments, high purification can be achieved with a single pass of the biofluid through the microchannel. It is anticipated that the methods are applicable to other classes of target cells with the use of appropriate antibodies.

It is noted that cocktails of antibodies, such as those included in the RosetteSep™ cell enrichment kits, are commonly used for cell separation in centrifugation processes. However, such cocktails generally include a wide variety of antibodies, many of which may not be necessary for the target separation. It is expected that in microfluidic separation processes, such as the methods disclosed herein, the wide variety of antibodies may not be as effective as a cocktail designed for the target separation. Thus, the methods disclosed herein employ a cocktail of antibodies specifically selected to bind the non-target cells in the biofluid. Purity and recovery may be increased by using an antibody cocktail selected, designed, or engineered to bind the selected non-target cells.

### Example 4: Enrichment of Selected Leukocytes from Blood Using a Cocktail of Antibodies Binding Non-Target Cells to Non-Target Cells

Improved enrichment of a target class of leukocytes from a blood sample was also achieved by introducing a cocktail of antibodies selected to bind a first class of non-target leukocytes to a second class of non-target leukocytes, forming the non-target cell clusters. The non-target cell-clusters were shown to be capable of being separated from the target leukocytes by acoustophoresis.

The target cells were selected to be NK cells and the antibodies were selected to bind non-target cells B cells, T cells, and monocytes, to deplete the non-target cells from the blood sample. The samples were prepared and treated generally as described in Example 3, except that the antibody cocktail was EasySep™ (distributed by StemCell Technologies, Vancouver, Calif.) at 25 μl/ml or 50 μl/ml. The EasySep™ product is conventionally used with magnetic beads for negative selection. However, here the antibody cocktail was introduced into leukapheresis product without addition of magnetic beads.

The results are shown in the graph of FIG. 17. The antibody cocktail increased NK cell purity from less than 20% in the initial leukapheresis sample (before incubation and separation) to 42% (25 μl/ml antibody cocktail) and 54% (50 μl/ml antibody cocktail) after incubation with the antibody cocktail and acoustophoretic separation.

Accordingly, significant purification was observed with the EasySep™ antibody cocktail and without the use of magnetic beads or dosing the sample with cluster-forming cells. It is hypothesized that the antibody labelled cells have an increased tendency to aggregate into clusters that are depleted by acoustophoresis.

### Example 5: Manufacture of Microfluidic Acoustophoresis Device

The device used in the previous examples was manufactured as described herein. Briefly, microchannels were fabricated from laminated sheets of general-purpose polystyrene, which was chosen for its prevalence in medical devices, optical clarity, and advantageous acoustic properties. The microfluidic channel (550 μm×250 μm×30 mm) and fluidic ports were precision-milled into the top layer of the polystyrene, which was then thermo-fusion bonded to a cover layer, for a total thickness of 2 mm After connecting medical grade tubing at the inlets and outlets, the channel was affixed with cyanoacrylate adhesive to a lead zirconate titanate (PZT) bulk transducer element, which generated the acoustic standing wave. The transducer was driven by conventional function generator and amplifier and monitored with an oscilloscope. A custom thermally-controlled aluminum stage was used as a platform to test the device at a constant 26° C. temperature using a temperature controller (distributed by Arroyo Instruments, San Luis Obispo, Calif.) and thermoelectric element (distributed by Laird Technologies, Inc., Chesterfield, Mo.).

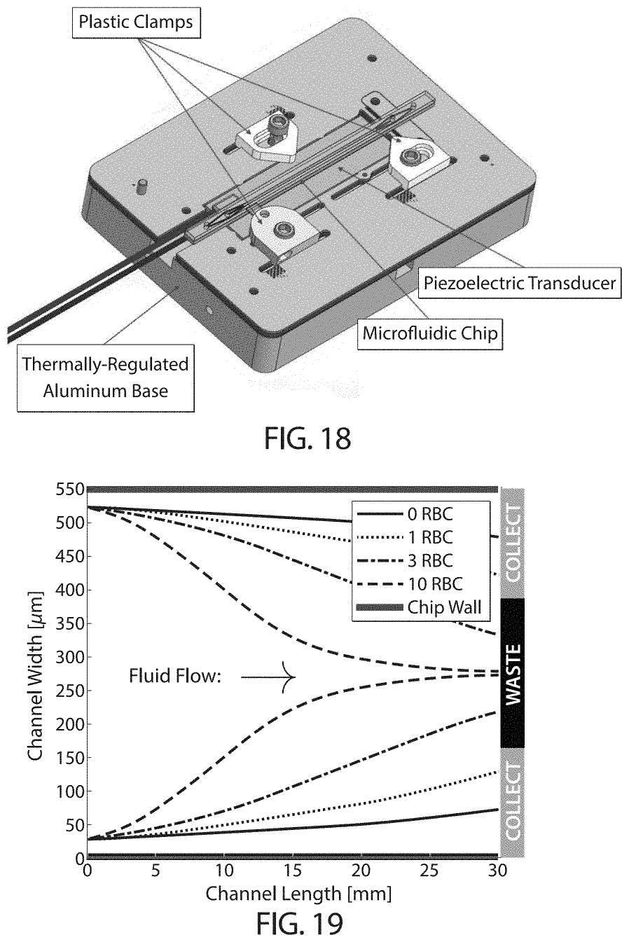

The mounting configuration is shown in the schematic drawing of FIG. 18. FIG. 18 is a rendering of the microfluidic acoustophoresis device, showing microfluidic channel (chip) mounted to piezoelectric transducer and clamped to thermal control stage. For clarity, the thermoelectric element and heat exchanger are not shown in FIG. 18.

Each device was calibrated in advance for its acoustophoretic performance by measuring the fraction of washed red blood cells that were focused toward the acoustic pressure node in the center of the channel at a given acoustic energy density, approximated by the voltage supplied to the transducer. During this calibration step, the optimal frequency of actuation (about 600-650 kHz) was tuned visually under bright field microscopy for each device. Because of variability among the devices used in these tests, the optimized transducer voltage and frequency varied between test runs. However, in each instance the calibration ensured that each device performed comparably. To ensure minimal deviation of device performance prior to the sample separations, calibrations and antibody treatments were completed consecutively.

### Example 6: Theoretical Separation

The mechanism of the observed purification and its dependence on the number of RBCs available can be further elucidated from a theoretical standpoint. Established theory provides a quantitative description of the acoustophoretic force on a spherical particle in a resonant rectangular microchannel and its resulting trajectory from inlet to outlet. As previously stated, the trajectory will generally depend on the size, density, and compressibility of the particle, as well as on other parameters of the device. The force on the particle toward the center axis of the channel scales with its volume, V, and acoustic contrast, Φ, in accordance with equation (1), where the contrast is a function of the particle's density and compressibility and that of the surrounding fluid.

F˜VΦ (1)

Adopting the simplified approximation of a one-dimensional channel cross section (i.e., infinitely shallow channel), and following generally accepted principles, the acoustic force along with the opposing velocity-dependent drag force due to the fluid can be integrated to obtain an expression for the displacement of the particle over time as it flows down the length of the channel in the acoustic field. With the further approximation that the residence time of the particle in the acoustic field is equivalent to the axial position z divided by the average fluid velocity flowing through the channel, a function for the trajectory of the particle in the plane is obtained and shown as equation (2), where α and β are constants of the acoustic device under fixed operating conditions, y₀ is the transverse position of the cell at the inlet, and r is the particle radius.

y(z)≈α arctan {tan [y₀/α] exp [βr²Φz]} (2)

For the cluster of cells, as opposed to a single spherical particle, the above analysis can be adapted taking:

$${V}^{′} = {V}_{1} + {V}_{2} + {V}_{3} … \qquad ( 3 )$$

$${Φ}^{′} = … \frac{{Φ}_{1} {V}_{1} + {Φ}_{2} {V}_{2} + {Φ}_{3} {V}_{3} …}{{V}^{′}} \qquad ( 4 )$$

$${r}^{′} = \sqrt[3]{\frac{3}{4 π} {V}^{′}} \qquad ( 5 )$$

In other words, V′ indicates the net volume of the cluster as the sum of the volumes of its component cells (indicated by the subscripts 1, 2, 3 . . . ), Φ′ an average of the contrast of the cells that make up the cluster, weighted by volume, and r′ an effective radius of the cluster. In this analysis, the main concern is the relative trajectories of unbound T cells (or target cells) compared with the trajectory of a non-target lymphocyte bound to some number of cells, optionally RBCs. The analysis is simplified because many constants of the device: the fluid, the flow rate, the acoustic energy, etc. can be normalized to any convenient value.

Using equation (2), with Φ′ substituted for Φ and r′ substituted for r, and fixing the constants α and β to achieve trajectories similar to the observed behavior, a highly simplified model of the impact of clusters on the separation of lymphocytes is obtained. FIG. 19 is a graph showing how the model can be used to investigate the significance of the number of RBCs bound to a lymphocyte. Values were obtained for the acoustic contrast of an average RBC in buffer and T-cell in buffer at diameters of 5.6 mm (spherical approximation) and 7 mm, respectively.

In the illustrated example, for a lymphocyte entering the channel at its upstream end and 25 mm from the channel sidewall, if unbound or bound to only one RBC, the lymphocyte will be collected at the side outlets. However, with 3 RBCs bound to the lymphocyte, the undesired lymphocyte is displaced by acoustophoresis to the waste outlet. With 10 RBCs bound to the lymphocyte, the cluster reaches nearly its terminal position at the channel center stream.

It is emphasized that the above calculations and the illustration of FIG. 19 are intended to provide a simplified view of cluster treatment in acoustophoresis. Many significant features of the real system were assumed to be negligent, including: velocity gradients in the fluid flow (Poiseuille flow), 3-dimensional non-uniformities in acoustic energy, and distribution of cell properties within each cell type. Additionally, treating the cluster of cells as a sphere of averaged properties does not take into account the effects of shape and heterogeneous compressibility of the cluster on the actual acoustophoretic force, which are still an area of active theoretical investigation. Despite these simplifications, the scaling suggests that the dependence on RBC concentration plotted in FIG. 15B may have room for improvement, including fewer RBCs or even no RBCs, as the method is further developed and the antibody cocktail is tailored specifically for the desired application.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed. For example, those skilled in the art may recognize that the method, and components thereof, according to the present disclosure may further comprise a network or systems or be a component of a system for microfluidic cell separation. It is therefore to be understood that the embodiments described herein are presented by way of example only and that, within the scope of the appended claims and equivalents thereto; the disclosed embodiments may be practiced otherwise than as specifically described. The present systems and methods are directed to each individual feature, system, or method described herein. In addition, any combination of two or more such features, systems, or methods, if such features, systems, or methods are not mutually inconsistent, is included within the scope of the present disclosure. The steps of the methods disclosed herein may be performed in the order illustrated or in alternate orders and the methods may include additional or alternative acts or may be performed with one or more of the illustrated acts omitted.

Further, it is to be appreciated that various alterations, modifications, and improvements will readily occur to those skilled in the art. Such alterations, modifications, and improvements are intended to be part of this disclosure and are intended to be within the spirit and scope of the disclosure. In other instances, an existing facility may be modified to utilize or incorporate any one or more aspects of the methods and systems described herein. Thus, in some instances, the systems may involve microfluidic cell separation. Accordingly, the foregoing description and figures are by way of example only. Further the depictions in the figures do not limit the disclosures to the particularly illustrated representations.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Disclosed aspects, or portions thereof, may be combined in ways not listed herein and/or not explicitly claimed. In addition, embodiments disclosed herein may be suitably practiced, absent any element that is not specifically disclosed herein. Accordingly, the invention should not be viewed as being limited to the disclosed embodiments.

While exemplary embodiments of the disclosure have been disclosed, many modifications, additions, and deletions may be made therein without departing from the spirit and scope of the disclosure and its equivalents, as set forth in the following claims.

What is claimed is:

1. A method of separating target cells from non-target cells in a biofluid, comprising:
pretreating the biofluid by introducing into the biofluid a predetermined amount of a cocktail of bifunctional antibodies selected to bind the non-target cells to form non-target cell clusters, producing a pretreated biofluid comprising the target cells and the non-target cell clusters;
flowing the pretreated biofluid into an inlet of a microfluidic separation channel; and
applying acoustic energy to the pretreated biofluid within the microfluidic separation channel, such that the target cells accumulate within at least one primary stream along the separation channel and the non-target cell clusters accumulate within at least one secondary stream along the separation channel.

2. The method of claim 1, wherein the bifunctional antibodies comprise at least one binding site having a non-specific affinity.

3. The method of claim 1, wherein pretreating the biofluid comprises introducing into the biofluid the predetermined amount of the cocktail of bifunctional antibodies without a capture particle.

4. The method of claim 1, further comprising selecting the biofluid from blood buffy coat, leukapheresis product, peripheral blood, whole blood, lymph fluid, synovial fluid, spinal fluid, bone marrow, ascities fluid, and combinations or subcomponents thereof.

5. The method of claim 1, further comprising selecting the target cells to be leukocytes selected from the group consisting of mononuclear cells, lymphocytes, monocytes, granulocytes, agranulocytes, macrophages, T cells, B cells, NK cells, subclasses thereof, and combinations thereof.

6. The method of claim 5, wherein the non-target cells comprise leukocytes selected from the group consisting of mononuclear cells, lymphocytes, monocytes, granulocytes, agranulocytes, macrophages, T cells, B cells, NK cells, subclasses thereof, and combinations thereof, other than the selected target cells.

7. The method of claim 6, comprising selecting the target cells to be lymphocytes.

8. The method of claim 1, further comprising selecting or designing the cocktail of the bifunctional antibodies responsive to a measured or expected cell population of the biofluid.

9. The method of claim 1, wherein the bifunctional antibodies have at least one binding site having affinity for a cluster-forming cell.

10. The method of claim 9, wherein the cluster-forming cells comprise erythrocytes or platelets.

11. The method of claim 9, further comprising controlling a ratio of the cluster-forming cells to the non-target cells in the biofluid.

12. The method of claim 11, further comprising introducing into the biofluid at least some of the cluster-forming cells.

13. The method of claim 1, further comprising obtaining the biofluid from a donor subject.

14. The method of claim 1, further comprising post-treating the at least one primary stream.

15. The method of claim 14, further comprising introducing the post-treated primary stream into a recipient subject.

16. The method of claim 1, further comprising flowing a second fluid adjacent to the biofluid into an inlet of the microfluidic separation channel, such that the biofluid and the second fluid flow in substantially parallel, substantially laminar flow.

17. The method of claim 1, further comprising flowing the pretreated biofluid into the inlet of the microfluidic separation channel at a flow rate of between about 0.03 mL/min to about 0.5 mL/min.

18. The method of claim 1, further comprising selecting the predetermined amount responsive to a parameter selected from input biofluid load, concentration of the target cells, concentration of the non-target cells, or concentration of cluster-forming cells of the biofluid.

19. The method of claim 18, further comprising measuring at least one of the input biofluid load, concentration of the target cells, concentration of the non-target cells, and concentration of the cluster-forming cells of the biofluid prior to pretreating the biofluid.

20. The method of claim 1, further comprising selecting a flow rate of the pretreated biofluid responsive to a parameter selected from pressure and acoustic energy within the microfluidic separation channel.

21. The method of claim 20, further comprising measuring at least one of pressure and acoustic energy within the microfluidic separation channel.

22. A system for microfluidic cell separation configured to separate target cells from non-target cells in a biofluid, comprising:
at least one microfluidic separation channel comprising at least one inlet, a first outlet, and a second outlet;
a source of the biofluid in fluid communication with the at least one inlet of the at least one microfluidic separation channel;
a source of an additive in fluid communication with the source of the biofluid, the additive comprising a cocktail of bifunctional antibodies selected to bind the non-target cells to form non-target cell clusters, producing a pretreated biofluid comprising the target cells and the non-target cell clusters; and
at least one acoustic transducer coupled to a wall of the at least one microfluidic separation channel.

23. The system of claim 22, wherein the additive is substantially free of capture particles.

24. The system of claim 22, further comprising a control module configured to introduce a predetermined volume of the additive into the biofluid to produce the pretreated biofluid.

25. The system of claim 22, wherein the at least one acoustic transducer is positioned to apply a standing acoustic wave transverse to the microfluidic separation channel.

26. The system of claim 22, comprising at least two microfluidic separation channels connected in parallel and a manifold configured to distribute the pretreated biofluid to the at least two microfluidic separation channels.

27. The system of claim 22, wherein the bifunctional antibodies comprise at least one binding site having a non-specific affinity.

28. The system of claim 22, wherein the bifunctional antibodies have at least one binding site having affinity for a cluster-forming cell.

29. The system of claim 28, further comprising a source of the cluster-forming cells in fluid communication with the source of the biofluid.

30. The system of claim 29, further comprising a control module in electrical communication with the source of the cluster-forming cells, configured to introduce a predetermined amount of the cluster-forming cells into the biofluid in response to a concentration of the cluster-forming cells and/or a concentration of the non-target cells in the biofluid.

31. A kit for microfluidic cell separation comprising:
at least one microfluidic separation channel comprising at least one inlet, a first outlet, and a second outlet;
a source of an additive fluidly connectable to the source of the biofluid, the additive comprising a cocktail of bifunctional antibodies selected to bind the non-target cells to form non-target cell clusters;
at least one acoustic transducer configured to be coupled to a wall of the at least one microfluidic separation channel; and
instructions to provide a biofluid, pretreat the biofluid by introducing a predetermined volume of the additive into the biofluid to form a pretreated biofluid comprising the target cells and the non-target cell clusters, flow the pretreated biofluid into the at least one inlet of the microfluidic separation channel, and apply acoustic energy to the microfluidic separation channel to separate the target cells from the non-target cell clusters.

32. The kit of claim 31, wherein the bifunctional antibodies comprise at least one binding site having a non-specific affinity.

33. The kit of claim 31, wherein the cocktail of the bifunctional antibodies is selected or designed responsive to a measured or expected cell population of the biofluid.

34. The kit of claim 31, wherein the additive is substantially free of capture particles.

35. A method of facilitating separation of target cells from non-target cells in a biofluid, comprising:
providing at least one microfluidic separation channel comprising at least one inlet, a first outlet, and a second outlet;
providing a source of an additive fluidly connectable to the source of the biofluid, the additive comprising a cocktail of bifunctional antibodies selected to bind the non-target cells to form non-target cell clusters;
providing at least one acoustic transducer configured to be coupled to a wall of the at least one microfluidic separation channel; and
providing instructions to pretreat the biofluid by introducing a predetermined volume of the additive into the biofluid to form a pretreated biofluid comprising the target cells and the non-target cell clusters, flow the pretreated biofluid into the at least one inlet of the microfluidic separation channel, and apply acoustic energy to the microfluidic separation channel to separate the target cells from the non-target cell clusters.

36. The method of claim 35, wherein the bifunctional antibodies comprise at least one binding site having a non-specific affinity.

37. The method of claim 35, wherein the cocktail of the bifunctional antibodies is selected or designed responsive to a measured or expected cell population of the biofluid.

38. The method of claim 35, comprising providing a control module configured to introduce the predetermined volume of the additive into the biofluid to produce the pretreated biofluid.

39. The method of claim 38, wherein the control module is configured to direct a pump to flow the pretreated biofluid into the at least one inlet of the microfluidic separation channel and direct the acoustic transducer to apply the acoustic energy to the microfluidic separation channel.

40. The method of claim 35, wherein the additive is substantially free of capture particles.
